(19) United States
(12) Patent Application Publication
(10) Pub. No.: US 2026/0207040 A1
(43) Pub. Date: Jul. 23, 2026

(54) DENTAL INSTRUMENT HAVING LIGHT SOURCES AND OPTICAL DIFFUSING ASSEMBLY AND METHOD OF USE THEREOF

(71) Applicant: HALO DENTAL TECHNOLOGIES INC., Brossard (CA)

(72) Inventors: Claude Chamoun, Brossard (CA); Peter EL KHOURY, Brossard (CA); Fadi KHOURY, Brossard (CA); Guillaume JOLY, Brossard (CA); Marc-Antoine ARCHAMBAULT, Montréal (CA); Mathieu BOUCHARD, Montréal (CA); David EL KHOURY, Saint-Lambert (CA); Rémy COUTURE, Saint-Basile-Le-Grand (CA)

(21) Appl. No.: 19/137,850

(22) PCT Filed: Dec. 12, 2023
(86) PCT No.: PCT/CA2023/051642

§ 371 Date: Jun. 11, 2025

Related U.S. Application Data
(60) Provisional application No. 63/387,027, filed on Dec. 12, 2022.

(51) Int. Cl. A61B 1/247; A61B 1/06; F21V 3/02; F21Y 115/10

(52) U.S. Cl. CPC A61B 1/247; A61B 1/06; A61B 1/0655; A61B 1/0684; F21V 3/02; F21Y 2115/10

(57) ABSTRACT

Dental instruments having light sources and methods of using such dental instruments are provided. The dental instrument can be configured for illuminating an oral cavity of a patient's mouth with diffused light, and can include a handle portion enabling the dental instrument to be handled, and a head portion positioned at one longitudinal end of the elongated member. The head portion can include a reflective layer that includes a diffused light guiding channel, an open-top housing defining a light source receiving cavity, light sources received within the light source receiving cavity, and an optical diffusing assembly that can be superposed to the light sources and that can be provided underneath the diffused light guiding channel, the optical diffusing assembly being configured to diffuse the light emitted by the light sources.

### TECHNICAL FIELD

The technical field generally relates to illuminating devices and systems. In particular, the technical field relates to dental instruments, such as dental mirrors, that include light sources for illuminating an oral cavity.

### BACKGROUND

It may be challenging for a health care provider, such as a dentist or a dental hygienist, to have a direct line of sight into a patient's mouth to perform a dental exam or other dental procedures. Intraoral mirrors, also known as dental mirrors, are frequently used by such health care providers to facilitate the viewing of biological structures that are located inside the oral cavity of the patient's mouth, including teeth and gums, by enabling the observation of reflections of the biological structure of interest onto the reflective surface of the dental mirror.

Nonetheless, an oral cavity of a patient's mouth is typically an environment that lacks sufficient illumination to properly view the biological structure of interest. Thus, in order to improve the visibility within the interior of an oral cavity of a patient's mouth, an external light source can be directed toward the oral cavity. For example, a bright external light source such as one that is focused via one or more reflective and/or lens elements, can be mounted on a multi-position armature to enable the health care provider to selectively position the external light source at a desired angle toward the oral cavity so as to provide illumination to a given region within the oral cavity of the patient's mouth.

However, such external light sources can have various drawbacks, such as inadequately illuminating the given region or biological structure in the oral cavity of the patient's mouth.

Accordingly, there remain a number of challenges with respect to dental instruments.

### SUMMARY

In accordance with an aspect, there is provided A dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   an open-top housing comprising a housing bottom wall, a housing sidewall having a housing sidewall outer surface, the housing bottom wall and the housing sidewall together defining a light source receiving cavity;
   light sources received within the light source receiving cavity and configured to emit light, the light sources being spaced-apart from one another and positioned according to a light source distribution; and
   an optical diffusing assembly superposed to the light sources and extending outwardly past outermost ones of the light sources in a direction away from a center of the light source receiving cavity, the optical diffusing assembly being configured to diffuse the light emitted by the light sources and produce the diffused light.

In some implementations, the optical diffusing assembly comprises an optical diffusing assembly sidewall having an outwardly oriented outer surface, the optical diffusing assembly sidewall and the housing sidewall together defining a head portion sidewall, and wherein the optical diffusing assembly is configured such that at least a portion of the diffused light travels away from the outwardly oriented outer surface, in a normal direction therefrom.

In some implementations, the optical diffusing assembly comprises:
an intermediate diffusing layer; and
an outer diffusing layer comprising the outwardly oriented outer surface of the optical diffusing assembly, the outer diffusing layer being provided furthest from the light sources compares to the intermediate diffusing layer;
wherein the intermediate diffusing layer and the outer diffusing layer are configured such that the light emitted from the light sources successively travels from the light sources, the intermediate diffusing layer and the outer diffusing layer.

In some implementations, the outer diffusing layer is an annular outer diffusing layer.

In some implementations, the intermediate diffusing layer has an intermediate diffusing layer surface area and the outer diffusing layer has an outer diffusing layer surface area, the outer diffusing layer surface area being smaller than intermediate diffusing layer surface area.

In some implementations, the outer diffusing layer is provided superposed to the intermediate layer so as to define a gap therebetween.

In some implementations, the outer diffusing layer is made of one or more of polystyrene, poly(methyl methacrylate), polycarbonate, and polypropylene.

In some implementations, the intermediate diffusing layer is made from of one or more of polystyrene, poly(methyl methacrylate), polycarbonate, and polypropylene.

In some implementations, the housing sidewall and the optical diffusing assembly sidewall are substantially aligned with one another to form a substantially continuous head portion sidewall.

In some implementations, the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

In some implementations, the light sources are provided at a regular interval from one another.

In some implementations, an upper portion of the optical diffusing assembly comprises a convex surface and a concave surface together defining an inwardly projecting edge.

In some implementations, the head portion further comprises a reflective layer superposed onto a portion of the optical diffusing assembly, the reflective layer having an outwardly oriented reflective surface.

In some implementations, the inwardly projecting edge is sized and configured so as to define a channel between the reflective layer and the inwardly projecting edge.

In some implementations, a cross-sectional width of the channel increases or remains constant from a lowest point of the channel to a top of the channel.

In some implementations, the reflective layer and at least a portion of optical diffusion assembly are configured to be removable from the head portion as a sub-assembly.

In some implementations, the head portion further comprises a printed circuit board comprising the light sources.

In some implementations, the head portion further comprises an optical detector, the optical detector being configured to:
detect at least one of an illuminance and a colour temperature;
compare at least one of:
   a detected illuminance with a target illuminance, and
   a detected colour temperature with a target colour temperature; and
   adjust a light output of the light sources based on the at least one comparison.

In some implementations, the light sources comprise light-emitting diodes.

In some implementations, the head portion further comprises an additional optical diffuser assembly, the additional optical diffuser assembly comprising:
additional light sources; and
an additional optical diffusing assembly defining at least part of the open-top housing.

In some implementations, the additional optical diffusing assembly comprises a diffusing layer received into diffusing layer openings defined in the housing bottom wall.

In some implementations, the second plurality of light sources comprise light-emitting diodes.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
a reflective layer having an outwardly oriented reflective surface;
   an open-top housing comprising a housing bottom wall comprising diffusing layer openings, a housing sidewall having a housing sidewall outer surface, the housing bottom wall and the housing sidewall together defining a light source receiving cavity;
   light sources received within the light source receiving cavity and configured to emit light in a direction opposite the outwardly oriented reflective surface of the reflective layer, the light sources being spaced-apart from one another and positioned according to a light source distribution; and
   an optical diffusing layer having an inwardly oriented surface facing the light sources and being received into the diffusing layer openings of the housing bottom wall, the optical diffusing layer being configured to diffuse the light emitted by the light sources and produce the diffused light.

In some implementations, the optical diffusing layer is made of one or more of polystyrene, poly(methyl methacrylate), polycarbonate, and polypropylene.

In some implementations, the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

In some implementations, the optical diffusing layer comprises a first optical diffusing layer and a second optical diffusing layer each being arcuate shaped.

In some implementations, the light sources are provided at a regular interval from one another.

In some implementations, the head portion further comprises a printed circuit board comprising the light sources.

In some implementations, the head portion further comprises an optical detector, the optical detector being configured to:
detect at least one of an illuminance and a colour temperature;
compare at least one of:
   a detected illuminance with a target illuminance, and
   a detected colour temperature with a target colour temperature; and
   adjust a light output of the light sources based on the at least one comparison.

In some implementations, the light sources comprise light-emitting diodes.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having a peripheral wall and an outwardly oriented reflective surface defined within the peripheral wall;
   an open-top housing comprising a housing bottom wall comprising diffusing layer openings, a housing sidewall having a housing sidewall outer surface, the housing bottom wall and the housing sidewall together defining a light source receiving cavity;
   light sources received within the light source receiving cavity and configured to emit light, the light sources being spaced-apart from one another and positioned according to a light source distribution such that at least outermost ones of the light sources are provided inwardly from the peripheral wall; and
   an optical diffusing assembly superposed to the light sources and extending outwardly from the peripheral wall of the reflective layer, the optical diffusing assembly being configured to diffuse the light emitted by the light sources and produce the diffused light.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   an open-top housing comprising a housing bottom wall a housing sidewall having a housing sidewall outer surface, the housing bottom wall and the housing sidewall together defining a light source receiving cavity;
   light sources received within the light source receiving cavity and configured to emit light, the light sources being spaced-apart from one another and distributed over an inwardly oriented surface area of the open-top housing; and
   an optical diffusing assembly extending at least between the light sources and the housing sidewall outer surface, the optical diffusing assembly being configured to diffuse the light emitted by the light sources and produce the diffused light.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having a peripheral wall and an outwardly oriented reflective surface defined within the peripheral wall;
   an open-top housing comprising a housing bottom wall, a housing sidewall having a housing sidewall outer surface, the housing bottom wall and the housing sidewall together defining a light source receiving cavity;
   light sources received within the light source receiving cavity and configured to emit light, the light sources being spaced-apart from one another; and
   an optical diffusing assembly superposed to the light sources and extending outwardly from the peripheral wall of the reflective layer, the optical diffusing assembly being configured to diffuse the light emitted by the light sources and produce the diffused light.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having an outwardly oriented reflective surface;
   an open-top housing comprising a housing bottom wall, a housing sidewall having a housing sidewall outer surface, the housing bottom wall and the housing sidewall together defining a light source receiving cavity;
   light sources received within the light source receiving cavity and configured to emit light in a direction opposite the outwardly oriented reflective surface of the reflective layer, the light sources being spaced-apart from one another and positioned according to a light source distribution.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having a peripheral wall and an outwardly oriented reflective surface defined within the peripheral wall;
   an open-top housing comprising a housing bottom wall comprising diffusing layer openings, a housing sidewall having a housing sidewall outer surface, the housing bottom wall and the housing sidewall together defining a light source receiving cavity;
   light sources received within the light source receiving cavity and configured to emit light, the light sources being spaced-apart from one another and positioned according to a light source distribution such that outermost ones of the light sources are provided at least partially outwardly from the peripheral wall of the reflective layer; and
      an optical diffusing assembly directly superposed to the light sources and extending outwardly from the peripheral wall of the reflective layer, the optical diffusing assembly being configured to diffuse the light emitted by the light sources and produce the diffused light.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having a peripheral wall and an outwardly oriented reflective surface defined within the peripheral wall;
   an open-top housing comprising a housing bottom wall, a housing sidewall having a housing sidewall outer surface, the housing bottom wall and the housing sidewall together defining a light source receiving cavity;
   light sources received within the light source receiving cavity and configured to emit light, the light sources being spaced-apart from one another; and
   an optical diffusing assembly directly superposed to the light sources and extending outwardly from the peripheral wall of the reflective layer, the optical diffusing assembly being configured to diffuse the light emitted by the light sources and produce the diffused light.

In accordance with another implementation, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having a peripheral wall and comprising a diffused light guiding channel extending peripherally along the peripheral wall and inwardly thereof;
   an open-top housing comprising a housing bottom wall and a housing sidewall together defining a light source receiving cavity;
   light sources received within the light source receiving cavity and configured to emit light; and
   an optical diffusing assembly superposed to the light sources and provided underneath the diffused light guiding channel, the optical diffusing assembly being configured to diffuse the light emitted by the light sources and produce the diffused light.

In some implementations, the light sources are spaced-apart from one another and positioned according to a light source distribution.

In some implementations, the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

In some implementations, the light sources are provided at a regular interval from one another.

In some implementations, the head portion further comprises a printed circuit board comprising the light sources.

In some implementations, the light sources comprise light-emitting diodes.

In some implementations, the optical diffusing assembly is directly superposed to the light sources and the diffused light guiding channel of the reflective layer is directly superposed to the optical diffusing assembly such that the light sources and the diffused light guiding channel share a corresponding common transversal axis extending normally relative to the housing bottom wall across the optical diffusing assembly.

In some implementations, the reflective layer comprises a transparent layer and a reflective surface positioned underneath the transparent layer, the optical diffusing assembly being positioned closest to the reflective surface.

In some implementations, the optical diffusing assembly comprises a single diffusing layer.

In some implementations, the optical diffusing assembly comprises a plurality of diffusing layers.

In some implementations, the optical diffusing assembly is sized to be contained within an outer periphery defined by the peripheral wall of the reflective layer.

In some implementations, the diffused light guiding channel coincides with the outer periphery defined by the peripheral wall.

In some implementations, the diffused light guiding channel is provided at a distance from the outer periphery defined by the peripheral wall, thereby defining a gap therebetween.

In some implementations, the diffused light guiding channel extends continuously at least from a 1 o'clock location to an 11 o'clock location.

In some implementations, the reflective layer further defines an optical detector opening at a 12 o'clock location.

In some implementations, the optical detector is configured to:
detect at least one of an illuminance and a colour temperature;
compare at least one of:
   a detected illuminance with a target illuminance, and
   a detected colour temperature with a target colour temperature; and
adjust a light output of the light sources based on the at least one comparison.
In some implementations, the optical diffusing assembly is substantially circular.
In some implementations, the optical diffusing assembly is ring-shaped and defines a diffusing layer opening.

In some implementations, the dental instrument further comprises an additional layer configured to be received in the diffusing layer opening.

In some implementations, the optical diffusing assembly defines an additional layer receiving surface configured to receive an additional layer thereon.

In some implementations, the additional layer is a heating layer.

In some implementations, the additional layer has a reduced ability to transmit light emitted by the light sources compared to a remainder of the optical diffusing assembly.

In some implementations, the additional layer is made of a light-blocking material.

In some implementations, the additional layer is made of a reflective material.

In some implementations, the optical diffusing assembly defines a fastener opening configured to receive a fastener therein to secure the optical diffusing assembly to the housing bottom wall.

In some implementations, the optical diffusing assembly is made of a diffusing material comprising one or more of polystyrene, poly(methyl methacrylate), polycarbonate, and polypropylene.

In some implementations, the diffusing material is configured to transmit between 25% and 90% of light having a wavelength between 400 nm and 800 nm.

In some implementations, the diffusing material has a density ranging between 1 000 kg/m³ and about 1 300kg/m³.

In some implementations, the optical diffusing assembly has a half-power angle ranging between about 1° and about 55°.

In some implementations, the optical diffusing assembly has a diffusing layer thickness ranging between about 0.5 mm and about 7 mm.

In some implementations, the optical diffusing assembly has a diffusing layer thickness of about 1 mm and a half-power angle of the optical diffusing assembly is between about 1° and about 5°.

In some implementations, the optical diffusing assembly has a diffusing layer thickness of about 2 mm and the half-power angle of the optical diffusing assembly is between about 18° and about 30°.

In some implementations, the optical diffusing assembly has a diffusing layer thickness of about 3 mm and the half-power angle of the optical diffusing assembly is between about 30° and about 45°.

In some implementations, the optical diffusing assembly has a diffusing layer thickness of about 4 mm and the half-power angle of the optical diffusing assembly is between about 40° and about 55°.

In some implementations, the head portion further comprises a head portion ring engageable with the open-top housing.

In some implementations, the head portion ring is screwable to a thread defined on the housing sidewall.

In some implementations, the head portion ring comprises an inwardly extending projection defining a reflective layer engaging surface for engaging with the peripheral wall of the reflective layer.

In some implementations, the reflective layer is shaped as a frustoconical reflective layer defining an outwardly extending slope configured to abut the reflective layer engaging surface of the inwardly extending projection of the head portion ring.

In some implementations, the dental instrument further comprises a gasket provided on a top edge of the housing sidewall, the gasket being compressible when subjected to a downward force as the head portion ring is being engaged with the open-top housing, thereby sealing the components of the dental instrument provided in the open-top housing.

In some implementations, the head portion ring and the reflective layer, and optionally the gasket, are configured to be removable from the head portion as a sub-assembly.

In some implementations, the reflective layer is a replaceable reflective layer.

In some implementations, the head portion further comprises an additional optical diffuser assembly, the additional optical diffuser assembly comprising:
additional light sources oriented opposite to the light sources; and
an additional optical diffusing assembly defining at least part of the housing bottom wall of the open-top housing.

In some implementations, the additional optical diffusing assembly comprises a diffusing layer received into diffusing layer openings defined in the housing bottom wall.

In some implementations, the additional light sources comprise light-emitting diodes.

In some implementations, the housing bottom wall defines a reflective layer receiving cavity, and the head portion further comprises a bottom reflective layer received in the reflective layer receiving cavity.

In some implementations, the housing bottom wall defines a step change at a transition between the housing bottom wall located outwardly of the bottom reflective layer and the reflective layer receiving cavity.

In some implementations, the dental instrument further comprises a gasket between a peripheral wall of the bottom reflective layer and the step change defined in the housing bottom wall.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   a reflective layer having an outwardly oriented reflective surface;
   an open-top housing comprising a housing sidewall and a housing bottom wall defining a diffusing layer opening, the housing bottom wall and the housing sidewall together defining a light source receiving cavity;
   light sources received within the light source receiving cavity and configured to emit light in a direction opposite the outwardly oriented reflective surface of the reflective layer; and
   an optical diffusing layer having an inwardly oriented surface facing the light sources and being received into the diffusing layer opening of the housing bottom wall, the optical diffusing layer being configured to diffuse the light emitted by the light sources and produce the diffused light.

In some implementations, the light sources are spaced-apart from one another and positioned according to a light source distribution.

In some implementations, the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

In some implementations, the optical diffusing layer comprises a first optical diffusing layer and a second optical diffusing layer and the diffusing layer opening comprises a first diffusing layer opening and a second diffusing layer opening, the first optical diffusing layer being received in the first diffusing layer opening and the second optical diffusing layer being received in the second diffusing layer opening, the first and second optical diffusing layers being shaped as semi-circular optical diffusing layers.

In some implementations, the head portion further comprises a printed circuit board comprising the light sources.

In some implementations, the light sources comprise light-emitting diodes.

In some implementations, the optical diffusing assembly is made of a diffusing material comprising one or more of polystyrene, poly(methyl methacrylate), polycarbonate, and polypropylene.

In some implementations, the diffusing material is configured to transmit between 25% and 90% of light having a wavelength between 400 nm and 800 nm.

In some implementations, the diffusing material has a density ranging between 1 000 kg/m³ and about 1 300kg/m³.

In some implementations, the optical diffusing assembly has a half-power angle ranging between about 1° and about 55°.

In some implementations, the optical diffusing assembly has a diffusing layer thickness ranging between about 0.5 mm and about 7 mm.

In some implementations, the optical diffusing assembly has a diffusing layer thickness of about 1 mm and a half-power angle of the optical diffusing assembly is between about 1° and about 5°.

In some implementations, the optical diffusing assembly has a diffusing layer thickness of about 2 mm and the half-power angle of the optical diffusing assembly is between about 18° and about 30°.

In some implementations, the optical diffusing assembly has a diffusing layer thickness of about 3 mm and the half-power angle of the optical diffusing assembly is between about 30° and about 45°.

In some implementations, the optical diffusing assembly has a diffusing layer thickness of about 4 mm and the half-power angle of the optical diffusing assembly is between about 40° and about 55°.

In some implementations, the head portion further comprises an optical detector, the optical detector being configured to:
detect at least one of an illuminance and a colour temperature;
compare at least one of:
   a detected illuminance with a target illuminance, and
   a detected colour temperature with a target colour temperature; and
adjust a light output of the light sources based on the at least one comparison.

In some implementations, the housing bottom wall defines a reflective layer receiving cavity, and the head portion further comprises a bottom reflective layer received in the reflective layer receiving cavity.

In some implementations, the housing bottom wall defines a step change at a transition between the housing bottom wall located outwardly of the bottom reflective layer and the reflective layer receiving cavity.

In some implementations, the dental instrument further comprises a gasket between a peripheral wall of the bottom reflective layer and the step change defined in the housing bottom wall.

In accordance with another aspect, there is provided a system for use with a head portion of a dental instrument, the system comprising:
a reflective layer comprising:
   a frustoconical transparent layer comprising a top surface, a bottom surface opposite the top surface, and a peripheral wall defining an outwardly extending slope; and
   a reflective surface adjacent to the transparent layer;
      wherein the peripheral wall of the reflective layer is configured to abut a reflective layer engaging surface of an inwardly extending projection of a head portion ring removably engageable with an open-top housing of the head portion, the reflective layer engaging surface and the outwardly extending slope having complimentary angles.

In some implementations, the reflective layer comprises a diffused light guiding channel extending along the peripheral wall of the transparent layer, inwardly thereof, the diffused light guiding channel being configured to enable passage of diffused light therethrough.

In some implementations, the system further comprising a gasket positionable underneath the reflective layer and configured to seal a remainder of the components of the head portion when a downward pressure is applied to the reflective layer.

In some implementations, the gasket is a compressible gasket.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   an open-top housing comprising a housing sidewall and a housing bottom wall defining a reflective layer receiving cavity, the housing sidewall and the housing bottom wall together defining a housing cavity;
   a top reflective layer at least partially received within the open-top housing; and
   a bottom reflective layer received in the reflective layer receiving cavity;
   wherein the top reflective layer and the bottom reflective layer are configured such that their respective reflective surfaces are opposite to each other.

In some implementations, the housing bottom wall defines a step change at a transition between the housing bottom wall located outwardly of the bottom reflective layer and the reflective layer receiving cavity.

In some implementations, the dental instrument further comprises a gasket between a peripheral wall of the bottom reflective layer and the step change defined in the housing bottom wall.

In some implementations, the top reflective layer comprises:
a frustoconical transparent layer comprising a top surface, a bottom surface opposite the top surface, and a peripheral wall defining an outwardly extending slope; and
a reflective surface adjacent to the transparent layer.

In some implementations, the head portion comprises a head portion ring configured to be removably engageable with the open-top housing.

In some implementations, the head portion ring comprises an inwardly extending projection defining a reflective layer engaging surface for engaging with the peripheral wall of the top reflective layer.

In some implementations, the peripheral wall of the top reflective layer is configured to abut the reflective layer engaging surface of the inwardly extending projection of the head portion ring, the reflective layer engaging surface and the outwardly extending slope having complimentary angles.

In some implementations, the top reflective layer comprises a diffused light guiding channel extending along the peripheral wall of the transparent layer, inwardly thereof, the diffused light guiding channel being configured to enable passage of diffused light therethrough.

In some implementations, the system further comprises a gasket positionable underneath the top reflective layer and configured to seal a remainder of the components of the head portion when a downward pressure is applied to the top reflective layer.

In some implementations, the gasket is a compressible gasket.

In accordance with another aspect, there is provided a dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
   an open-top housing comprising a housing bottom wall and a housing sidewall together defining a light source receiving cavity;
   light sources received within the light source receiving cavity and configured to emit light; and
   a reflective layer having a peripheral wall and comprising a diffused light guiding channel superposed to the light sources and extending peripherally along the peripheral wall and inwardly thereof, the diffused light guiding channel comprising:
   an optical diffusing assembly configured to diffuse the light emitted by the light sources and produce the diffused light.

### BRIEF DESCRIPTION OF THE DRAWINGS

The attached figures illustrate various features, aspects and implementations of the technology described herein.

FIG. 1 is a perspective view of a dental instrument that includes a head portion and a handle, according to an implementation.

FIG. 2 is a top plan view of the head portion of the dental instrument of FIG. 1.

FIG. 3 is bottom plan view of the head portion of the dental instrument of FIG. 1.

FIG. 4 is a cross-sectional perspective view of the head portion of the dental instrument of FIG. 1.

FIG. 5 is a cross-sectional side view of an enlarged section of the head portion of the dental instrument of FIG. 1.

FIG. 6 is a cross-sectional front view of the head portion of the dental instrument of FIG. 1.

FIG. 7 is a perspective view of an open-top housing of the head portion of the dental instrument of FIG. 1.

FIG. 8 is another perspective view of the open-top housing of FIG. 7.

FIG. 9 is a cross-sectional side view of the open-top housing of FIG. 7.

FIG. 10 is a top perspective view of a printed circuit board receivable within the open-top housing of the head portion of the dental instrument of FIG. 1.

FIG. 11 is a top plan view of the printed circuit board of FIG. 10.

FIG. 12 is a bottom perspective view of the printed circuit board of FIG. 10.

FIG. 13 is a bottom plan view of the printed circuit board of FIG. 10.

FIG. 14 is a top perspective view of an intermediate diffusing layer of the dental instrument of FIG. 1.

FIG. 15 is a top plan view of the intermediate diffusing layer of FIG. 14.

FIG. 16 is a bottom perspective view of the intermediate diffusing layer of FIG. 14.

FIG. 17 is a bottom plan view of the intermediate diffusing layer of FIG. 14.

FIG. 18 is a top perspective view of an outer diffusing layer of the dental instrument of FIG. 1.

FIG. 19 is a cross-sectional perspective view of the outer diffusing layer of FIG. 18.

FIG. 20 is a top plan view of the outer diffusing layer of FIG. 18.

FIG. 21 is a bottom plan view of the outer diffusing layer of FIG. 18.

FIG. 22 is a perspective view of optical diffusing layers configured to be received in diffusing layer openings defined in a bottom wall of the open-top housing of the head portion of the dental instrument of FIG. 1.

FIG. 23 is a partially exploded, partially cross-sectional, perspective view of a head portion of the dental instrument of FIG. 1, with the outer diffusing layer and the reflective layer shown separated from the head portion.

FIG. 24 is a perspective view of the head portion of FIG. 23, and of a tool configured to facilitate separation of the outer diffusing layer and the reflective layer from the head portion.

FIG. 25 is a perspective view of a dental instrument that includes a head portion and a handle portion, according to another implementation.

FIG. 26 is a front perspective view of the dental instrument shown in FIG. 25.

FIG. 27 is a top perspective view of the dental instrument shown in FIG. 25.

FIG. 28 is an enlarged cross-sectional view of the head portion of the dental instrument shown in FIG. 25 taken along line A shown in FIG. 26.

FIG. 29 is a cross-sectional view of the head portion of the dental instrument shown FIG. 28.

FIG. 30 is an enlarged cross-sectional view of the head portion of the dental instrument shown in FIG. 25 taken along line C shown in FIG. 27.

FIG. 31 is a cross-sectional view of the head portion of the dental instrument shown FIG. 30.

FIG. 32 is a bottom exploded view of the dental instrument shown in FIG. 25.

FIG. 33 is a top exploded view of the dental instrument shown in FIG. 25.

### DETAILED DESCRIPTION

Techniques described herein relate to systems, devices and methods for illuminating an oral cavity of a patient's mouth with diffused light in the context of various procedures that can be performed for instance by health care providers, such as a dental exam, a cavity repair, etc. Illuminating the patient's mouth with diffused light can be achieved using a dental instrument as described herein. The dental instrument can be manipulated by the health care provider, and a portion of the dental instrument can be configured to be introduced into the oral cavity of the patient's mouth. More particularly, the dental instrument can include an elongated member enabling the dental instrument to be handled by the health care provider, and a head portion that is positioned at one longitudinal end of the elongated member and that is configured to be introduced into the oral cavity of the patient's mouth. The head portion can include an open-top housing, light sources and an optical diffusing assembly to produce diffused light. The open-top housing includes a housing bottom wall, a housing sidewall having a housing sidewall outer surface, the housing bottom wall and the housing sidewall together defining a light source receiving cavity. The light sources are received within the light source receiving cavity and configured to emit light, the light sources being spaced-apart from one another and positioned according to a light source distribution. The optical diffusing assembly is superposed to the light sources. In some implementations, the optical diffusing assembly can be configured to extend outwardly past outermost ones of the light sources in a direction away from a center of the light source receiving cavity. The optical diffusing assembly, at least because of the material from which it is made of, is configured to diffuse the light emitted by the light sources and produce the diffused light.

In some implementations, the dental instrument can further include a reflective layer, e.g., a mirror, so that the dental instrument can be used as a dental mirror. Health care providers, such as dentists and dental hygienists, are trained to work with an indirect view of an area of interest within the oral cavity, while handling an operating light that is located outside of the oral cavity of the patient's mouth. Handling an operating light that is located outside of the oral cavity of the patient's mouth requires the health care provider to interrupt the procedure being performed to adjust the positioning of the operating light, increasing the time of the procedure. Furthermore, an operating light that is located outside of the oral cavity of the patient's mouth may inefficiently illuminate the area of interest. In addition, although dental mirrors with discrete sources of light distributed around the mirror surface may be used, this type of dental mirrors has several drawbacks, such as producing intense and uneven light spots that may distract the health care provider, produce glare, and inefficiently illuminate the area of interest. Providing a dental instrument, such as a dental mirror, as described herein that produces diffused light to illuminate an area of interest within the oral cavity can provide various benefits over an operating light that is located outside of the oral cavity of the patient's mouth that may not be suitable to sufficiently illuminate the area of interest. The dental instrument can include one or more optical diffusing assembly. When one optical diffusing assembly is present, the optical diffusing assembly can be located on a front of the head portion of the dental instrument or a rear of the head portion of the dental instrument. When two optical diffusing assemblies are present, a first optical diffusing assembly can be located on the front of the head portion of the dental instrument and a second optical diffusing assembly can be located on the rear of the head portion of the dental instrument.

Alternatively, the dental instrument may not include a reflective layer, and may otherwise include another type of instrument at a longitudinal end of the elongated member, or the dental instrument can be used as a source of light, without any other additional instruments being present.

In some implementations, the light sources provided within the head portion of the dental instrument can be configured to automatically adjust their light output based on detected illumination levels. For example, the dental instrument can include an optical detector, and the detect can be configured to detect an illuminance and/or a colour temperature, and adjust the light output based on a comparison of the detected illuminance and/or colour temperature with one or more target values.

It is to be noted that while the systems, devices and methods described herein are presented in the context of dental instruments that can be used during dental procedures typically performed on humans, it will be appreciated that the systems, devices and methods can alternatively be used in the context of veterinary treatments. In addition, the systems, devices and methods described herein can also be used in other contexts when illumination of a biological cavity that is not an oral cavity, or of a cavity that is not a biological cavity.

Various implementations and features of the dental instrument and associated methods will now be described in greater detail in the following paragraphs.

### General Description of Dental Instrument

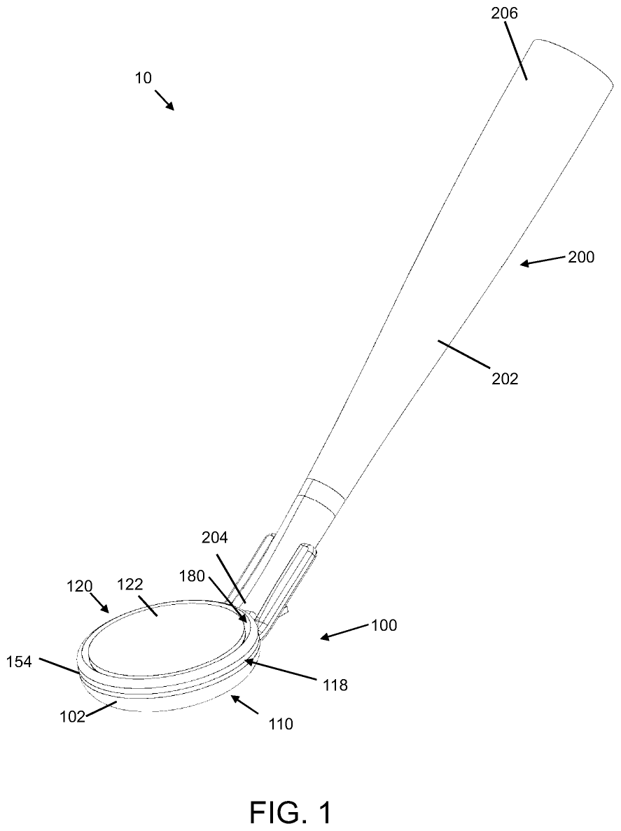

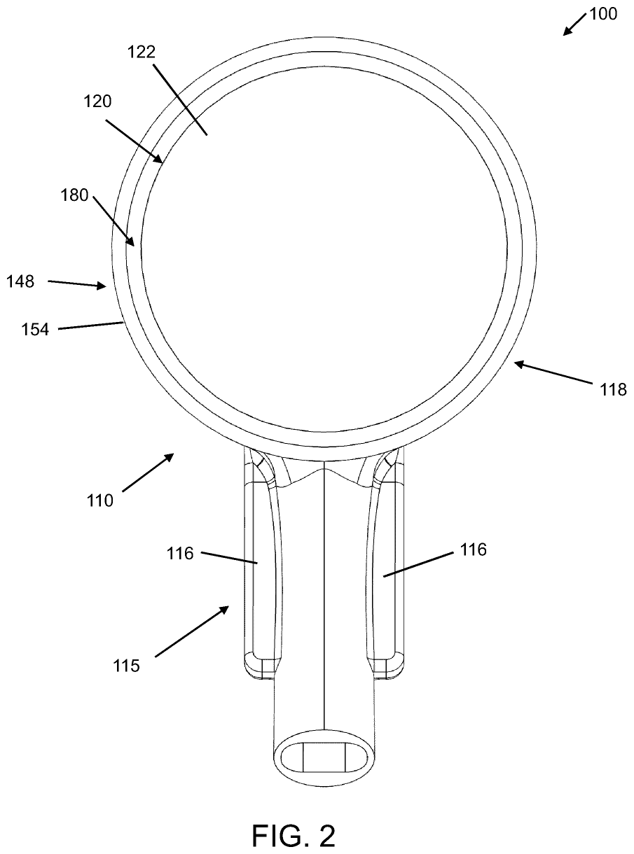

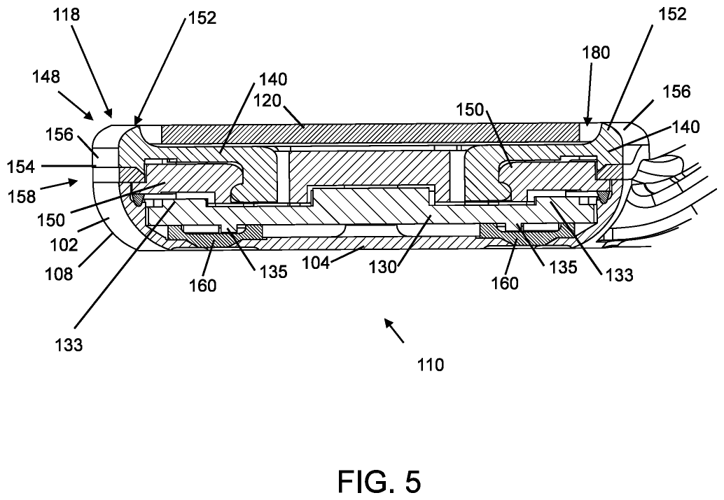

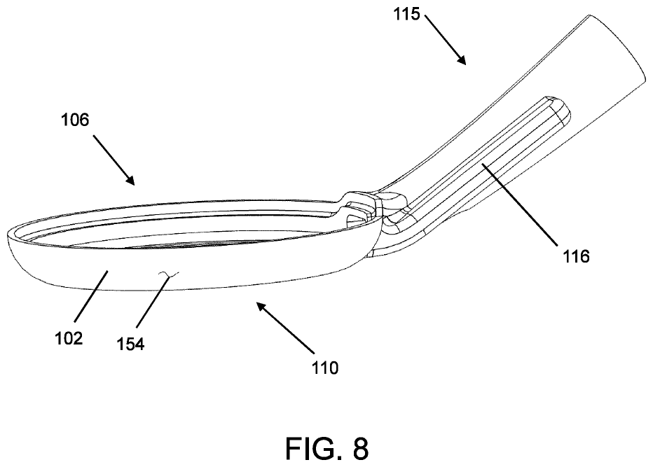

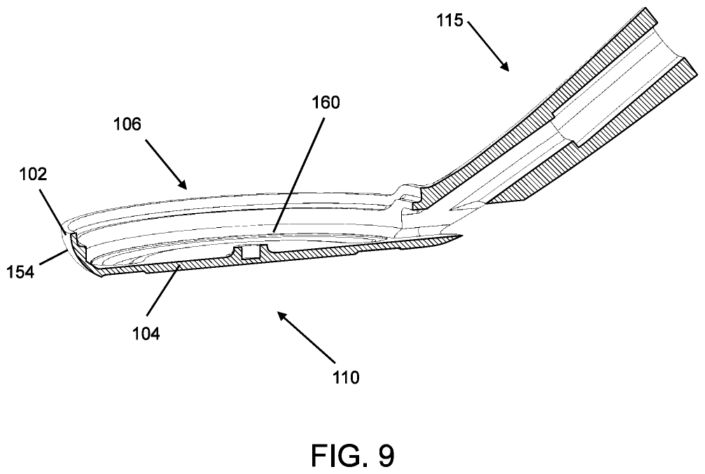

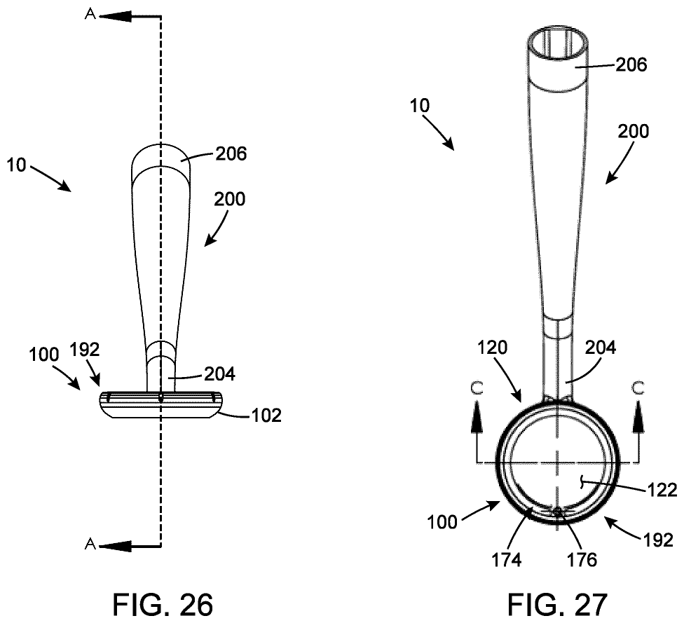

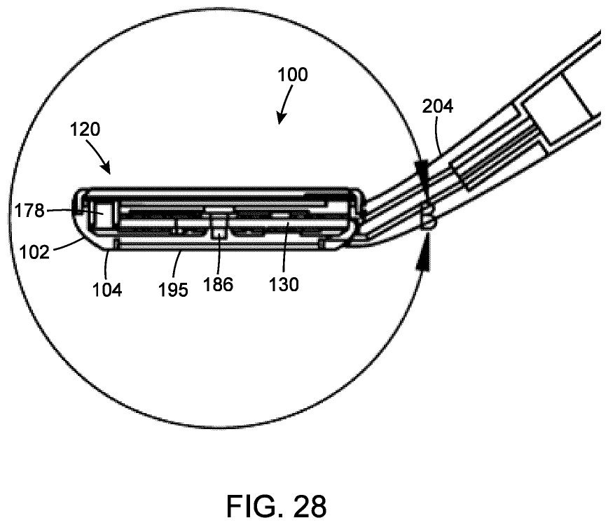

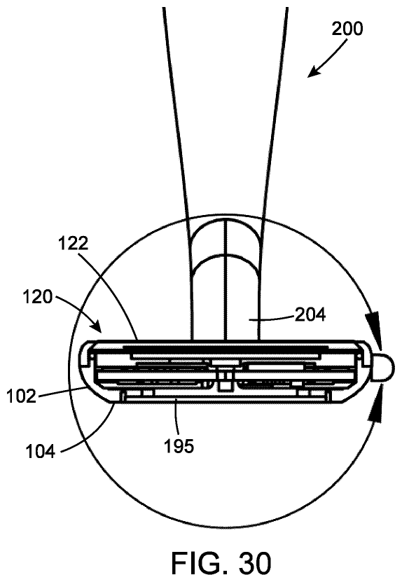

With reference to FIGS. 1 to 9 and 25 to 33, implementations of a dental instrument 10 is shown. The dental instrument 10 includes a handle portion 200 that includes an elongated member 202 having a first longitudinal end 204 and a second longitudinal end 206, the second longitudinal end 206 being located opposite to the first longitudinal end 204. The dental instrument 10 further includes a head portion 100 positioned at, or in proximity to, the first longitudinal end 204 of the elongated member 202. In the implementation shown, the head portion 100 of the dental instrument 10 includes a reflective layer 120 having a reflective surface 122. The reflective layer 120 can be for instance a mirror, or other types of wave reflector. The reflective surface 122 may be located on the back (for instance forming a back-silvered mirror) or in the front (for instance forming a front-silvered mirror). When the dental instrument includes the reflective layer 120, a health care provider can grasp the elongated member 202 of the handle portion 200 to manipulate the position of the head portion 100, e.g., within an oral cavity of a patient's mouth, relative to various biological structures of interest in the oral cavity in order to indirectly view of a given area as reflected by the reflective surface 122 of the reflective layer 120. In some implementations, the reflective layer 120 can be omitted, and be replaced by a layer that is not reflective, to achieve a similar configuration of the head portion 100 as described herein but without having the reflective surface being present.

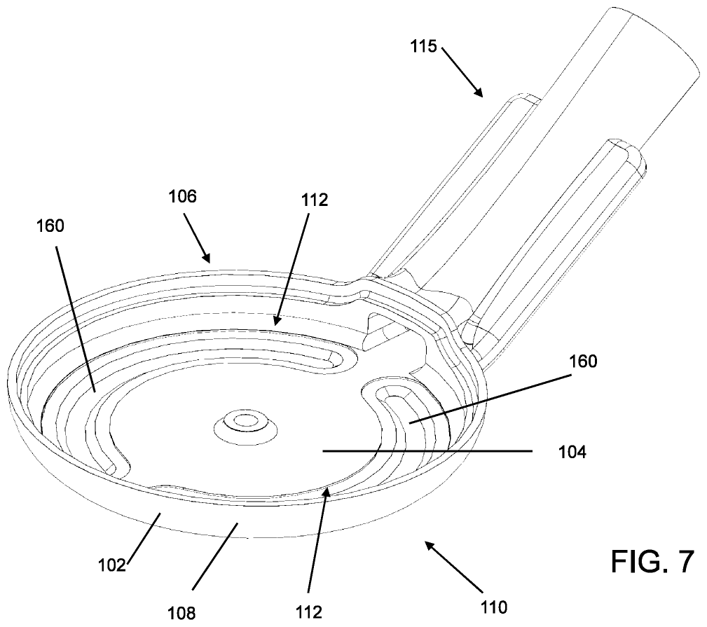

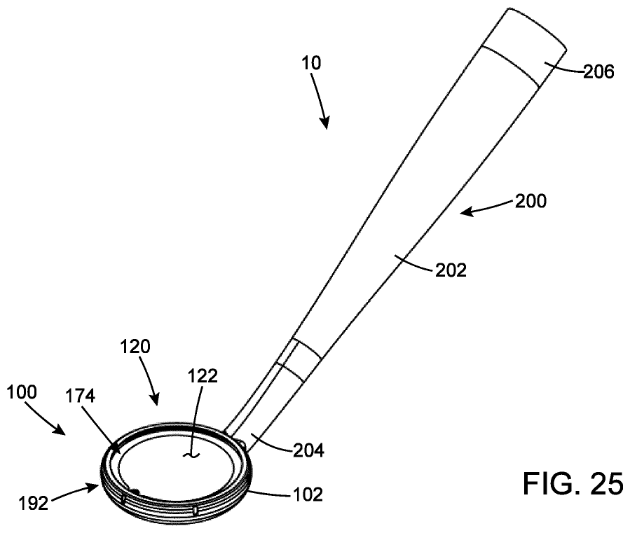

The head portion 100 includes an open-top housing 110. With reference more particularly to FIGS. 7 to 9, 32 and 33, the open-top housing 110 includes a housing sidewall 102 and a housing bottom wall 104. The housing sidewall 102 includes a housing sidewall outer surface 108. The combination of the housing sidewall 102 and the housing bottom wall 104 together define a light source receiving cavity 106. In implementation illustrated in FIGS. 7 to 9, the open-top housing 110 includes an elongated member engaging stem 115 for engaging the head portion 100 with the elongated member 202 of the handle portion 200 at a given relative angle. The elongated member engaging stem 115 can be integral with the open-top housing 110, such as shown in the implementation illustrated in FIGS. 1 to 9. In other implementations, the elongated member engaging stem 115 can be removably engageable with the head portion 100, or connected to the open-top housing in any other suitable way. Furthermore, in the implementation shown in FIGS. 1 to 9, the elongated member engaging stem 115 includes a pair of stem wings 116 provided on each side thereof, to strengthen the connection between the elongated member engaging stem 115 and the open-top housing 110. It is to be understood that these stem wings 116 are optional and may be omitted, as shown in FIGS. 25 to 33.

In the implementations shown, the housing sidewall 102 is substantially cylindrical, with the housing bottom wall 104 having a substantially circular surface area. This combination of the housing sidewall 102 and the housing bottom wall 104 results in the open-top housing 110 being shaped like a cup, or a saucer, with the housing sidewall 102 curving inwardly such that the diameter of the housing bottom wall 104 is smaller than the diameter at the top of the open-top housing 110. In other implementations, the housing sidewall 102 can be substantially straight, such that the diameter of the housing bottom wall 104 and the diameter at the top of the open-top housing 110 are substantially similar. It is to be understood that the housing sidewall 102 and the housing bottom wall 104 can have different shapes than the one exemplified in FIGS. 1 to 9 and 25 to 33, depending for instance on the configuration of the remainder of the dental instrument 10 and its intended use. In implementations where the dental instrument 10 is intended to be used to illuminate the oral cavity of a patient's mouth, providing the open-top housing 110 with a housing sidewall 102 that is edgeless, or curvilinear, can contribute to increasing the comfort of the patient once the head piece 100 is introduced in the oral cavity.

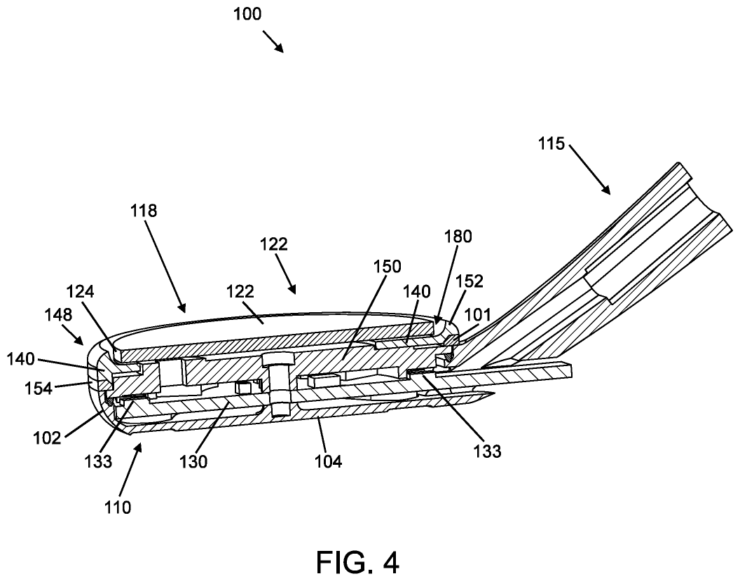

With reference to FIGS. 4 to 6, 29, 31 and 33, the head portion 100 includes light sources 133 received within the light source receiving cavity 106 of the open-top housing 110, the light sources 133 being configured to emit light. The light sources 133 are spaced-apart from one another, and positioned according to a light source distribution. More details regarding the light source distribution will be provided below.

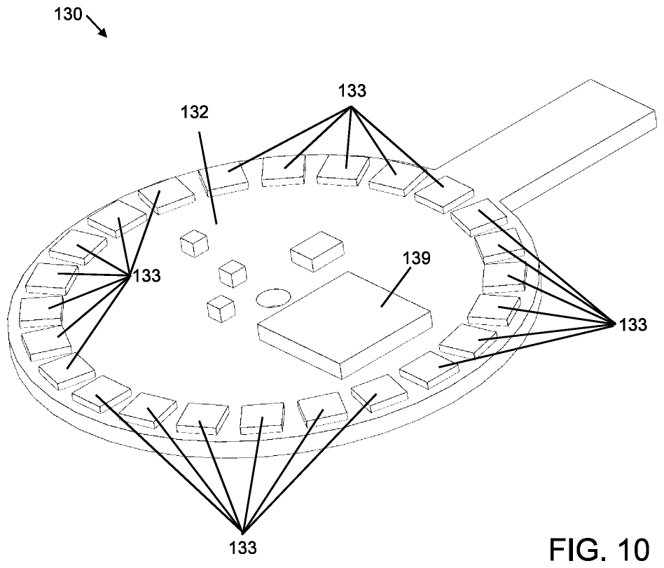

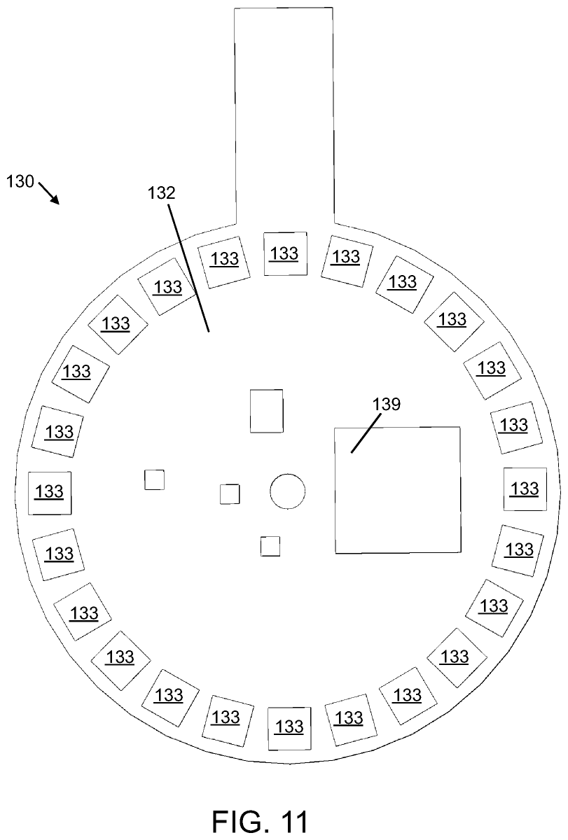

The light sources 133 are configured and oriented to emit light upwardly and away from the housing bottom wall 104, e.g., toward the underside of the reflective layer 120 when the reflective layer 120 is present. In the implementation shown, the light sources 133 are provided as components of a printed circuit board (PCB) 130 received within the light source receiving cavity 106. The printed circuit board 130 also further includes additional light sources 135 that are provided on a lower surface 134 of the printed circuit board 130. In some implementations, the light sources 133 and the additional light sources 135 can include light-emitting diodes (LEDs). In other implementations, any other types of light sources that are configured to emit light can also be suitable. Furthermore, although no light sources are illustrated in a center region of the printed circuit board 130 in FIGS. 10 to 12 and 33, in other implementations, light sources can be present in the center region of the printed circuit board 130.

The head portion 100 further includes an optical diffusing assembly 118. The optical diffusing assembly 118 is provided between the printed circuit board 130 and the reflective layer 102, and is thus partially received within the light source receiving cavity 106 of the open-top housing 110.

In some implementations, and as shown in FIGS. 4 to 6, the optical diffusing assembly 118 includes an outer diffusing layer 140 and an intermediate diffusing layer 150. The intermediate diffusing layer 150 is provided closest to the light sources 133, and is thus “sandwiched” between the outer diffusing layer 140 and the light sources 133. The optical diffusing assembly 118 includes an optical diffusing assembly sidewall 154 having an outwardly oriented outer surface 156. Accordingly, when the optical diffusing assembly 118 includes an outer diffusing layer 140 and an intermediate diffusing layer 150, the light emitted from the light sources 133 can successively travel from the light sources 133, the intermediate diffusing layer 150 and the outer diffusing layer 140. In the implementation shown in FIGS. 4 to 6, the outer diffusing layer 140 is provided superposed to the intermediate diffusing layer 150 so as to define a gap therebetween. It is to be noted that in other implementations, the gap can be omitted, such that the outer diffusing layer 140 can be in direct contact with the intermediate diffusing layer 150.

As can be seen in FIGS. 4 to 6, for example, the combination of the housing sidewall 102 and the optical diffusing assembly sidewall 154 together form the sidewall of the head portion 100, i.e., a head portion sidewall 158. In the implementation illustrated in FIGS. 4 to 6, the combination of the housing sidewall 102 and the optical diffusing assembly sidewall 154 are shown such that the housing sidewall outer surface 108 and the outwardly oriented outer surface 156, respectively, are substantially aligned with each other. It is to be understood that in other implementations, the housing sidewall 102 and the optical diffusing assembly sidewall 154 can be configured so as to be offset from each other, with the optical diffusing assembly sidewall 154 being provided either inwardly or outwardly relative to the housing sidewall 102.

Referring to FIGS. 25 to 33, in other implementations, the optical diffusing assembly 118 itself can designate a single diffusing layer provided between the light sources 133 and the reflective layer 102, and the optical diffusing assembly 118 can be at least partially received within the light source receiving cavity 106 of the open-top housing 110. Alternatively, the optical diffusing assembly 118 can include a plurality of diffusing layers configured as shown in FIGS. 4 to 6 or configured differently than exemplified in FIGS. 4 to 6.

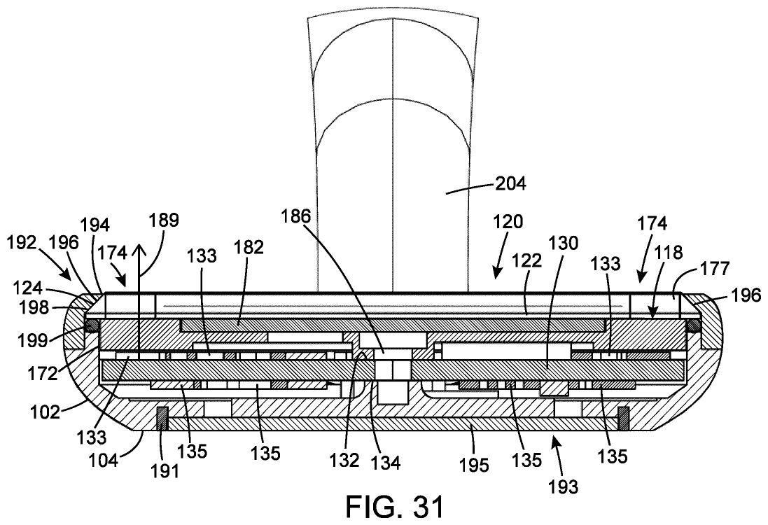

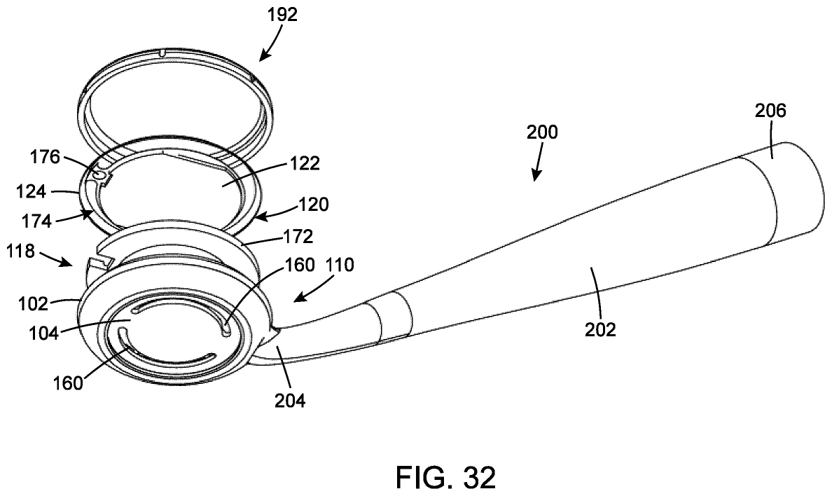

In the implementation shown in FIGS. 25 to 33, the optical diffusing assembly 118 is located underneath the reflective layer 120, such that the outer periphery 172 of the optical diffusing assembly 118 is sized so as to be contained within the outer periphery defined by the peripheral wall 124 of the reflective layer 120. In other words, the width of the optical diffusing assembly 118, or diameter when the optical diffusing assembly 118 is circular, is equal or less than the width of the reflective layer 120. In such implementations, and as illustrated more particularly in FIGS. 31 to 33, the reflective layer 120 comprises a diffused light guiding channel 174 located inwardly of the peripheral wall 124 of the reflective layer 120. The diffused light guiding channel 174 extends peripherally along the peripheral wall 124, or circumferentially along the peripheral wall 124 when the reflective layer 120 is circular. The diffused light guiding channel 174 can thus follow the same profile as the peripheral wall 124 of the reflective layer 120, and can be substantially circular when the reflective layer 120 is circular. It is to be understood that when the reflective layer 120 comprises a diffused light guiding channel 174, the width of the optical diffusing assembly 118 can be larger than the width of the reflective layer 120, and thus can extend outwardly past the peripheral wall 124 of the reflective layer 120, depending on the position of the other remaining components of the head portion 100 of the dental instrument 10.

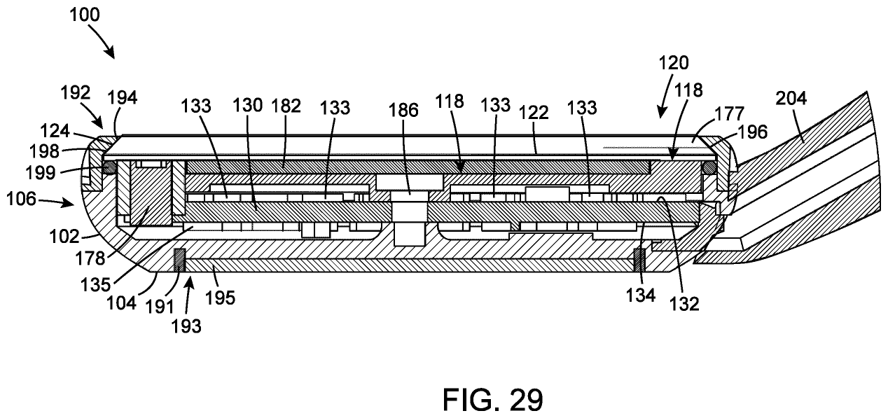

In the implementation shown in FIGS. 25 to 33, the diffused light guiding channel 174 is discontinuous at a location corresponding to approximately 12 o'clock of the reflective layer 102, where an optical detector opening 176 is defined. Accordingly, in this implementation, the diffused light guiding channel 174 can be described as extending continuously at least from a 1 o'clock location to an 11 o'clock location. The optical detector opening 176 can be configured to enable an optical detector located within the light source receiving cavity 106 to capture images through the optical detector opening 176. Accordingly, when an optical detector is present, one or more of the light sources 133 can be omitted at the location of the optical detector. FIG. 29 illustrates an example of an optical detector 178 received within the light source receiving cavity 106. In other implementations, the diffused light guiding channel 174 can be discontinuous at other locations as well, to form a plurality of circular arcs.

In the implementation shown, the diffused light guiding channel 174 extends outwardly to reach the peripheral wall 124 of the reflective layer 120, such that the diffused light guiding channel 174 substantially coincides with the outer periphery of the peripheral wall 124 of the reflective layer 120. The width of the diffused light guiding channel 174 can be chosen to enable sufficient diffused light to travel out of the reflective layer 120 and light up the inside of the oral cavity as desired by the health care provider. In other implementations, there can be a gap between the peripheral wall 124 of the reflective layer 120 and the outer periphery of the diffused light guiding channel 174.

In implementations where the reflective layer 120 comprises a diffused light guiding channel 174, the reflective layer 120 can include a transparent layer 177 made of a transparent material, such as glass or sapphire, and a reflective surface 122 provided in a center region of the reflective layer 120. The diffused light guiding channel 174 can be defined through the thickness of the transparent layer 177, and can be delimited inwardly by the outline of the reflective surface 122. Accordingly, the diffused light from the optical diffusing assembly 118 can exit the optical diffusing assembly 118 and travel upwardly through the thickness of the transparent layer 177. In some implementations, the diffused light can thus be produced by the optical diffusing assembly 118 and travel out of the reflective layer 120 via the diffused light guiding channel 174 without substantially being further diffused. In the illustrated implementation, the reflective surface 122 is provided underneath the transparent layer 177, although other configurations are also possible. In alternative implementations, the diffused light guiding channel 174 can also be configured to contribute to further diffuse the diffused light from the optical diffusing assembly 118. Further details regarding the configuration of the diffused light guiding channel 174 when the diffused light guiding channel 174 is configured to diffuse light are provided below.

The optical diffusing assembly 118 is configured to diffuse light emitted by the light sources 133 and produce diffused light. In order to do so, the optical diffusing assembly 118 can be made of a diffusing material that is configured to scatter light, such as collimated light, from the light sources 133 to transmit a soft, substantially uniform and sober light, instead of many discrete “point sources” that could otherwise be transmitted by the light sources 133, in the absence of the optical diffusing assembly 118. In other words, the optical diffusing assembly 118 can be made of a diffusing material that is configured to scatter light such that its spatial coherence is substantially reduced. Such many discrete “point sources” of light can be undesired as they can be distracting to the health care provider handling the dental instrument 10, can produce glare, and can also provide uneven illumination to an area of interest within the oral cavity. In some implementations, the optical diffusing assembly 118 is configured to create Lambertian scattering where the radiance is independent of angle. The optical diffusing assembly 118 can thus be made of any suitable diffusing material, such as translucent polymers.

In the present description, the term “transparent” refers to the capability of a material to allow electromagnetic radiation in a certain spectral region to pass therethrough without appreciable scattering. The term “translucent” refers to the capability of a material to allow electromagnetic radiation in a certain spectral region to pass therethrough with appreciable scattering. As mentioned above, in some implementations, the diffusing material can be made of a translucent polymer. In some implementations, the diffusing material can be for instance one or more of polystyrene, poly(methyl methacrylate), acrylic, glass, polycarbonate, and polypropylene. In some implementations, the diffusing material can be made of polysiloxane, polymethylsilsesquioxane or silicone. It is to be understood that any material having the capability to diffuse light, i.e., scatter light, can be a suitable material for the optical diffusing assembly 118.

In some implementations, the optical diffusing assembly 118 can be made of a diffusing material having a density ranging between 1 000 kg/m³ and about 1 300kg/m³.

In some implementations, the optical diffusing assembly 118, when present as a single diffusing layer, can have a diffusing layer thickness ranging between about 0.5 mm and about 7 mm.

In some implementations, the optical diffusing assembly 118 can be made of a diffusing material that is configured to transmit between 25% and 90% of light having a wavelength between 400 nm and 800 nm.

In some implementations, the optical diffusing assembly 118 can be made of a diffusing material that is configured to diffuse light such that light transmitted through the optical diffusing assembly 118 is scattered according to a half-power angle ranging between about 1° and about 55°. The half-power angle is a measure that can be used to evaluate the degree of scattering of light passing through a diffuser, such as the optical diffusing assembly described herein. To measure the half-power angle, collimated light is impinged on one side of a sample and the light intensity (luminance) is measured as a function of angle on the other side of the sample. When the luminance is plotted as a function of angle, a narrow beam spread can be interpreted as corresponding to a lower diffusion and a wide beam spread can be interpreted as corresponding to a higher diffusion. The half-power angle is the angle at which the light intensity decreases to half of its maximum value. A similar approach is used when measuring the Full Width Half Maximum (FWHM), which corresponds to twice the half-power angle.

In some implementations, the optical diffusing assembly can have a diffusing layer thickness of about 1 mm, and have a half-power angle of between about 1° and 5°. In some implementations, the optical diffusing assembly can have a diffusing layer thickness of about 2 mm, and have a half-power angle between about 18° and about 30°. In some implementations, the optical diffusing assembly can have a diffusing layer thickness of about 3 mm, and have a half-power angle between about 30° and about 45°. In some implementations, the optical diffusing assembly can have a diffusing layer thickness of about 4 mm, and have a half-power angle between about 40° and about 55°.

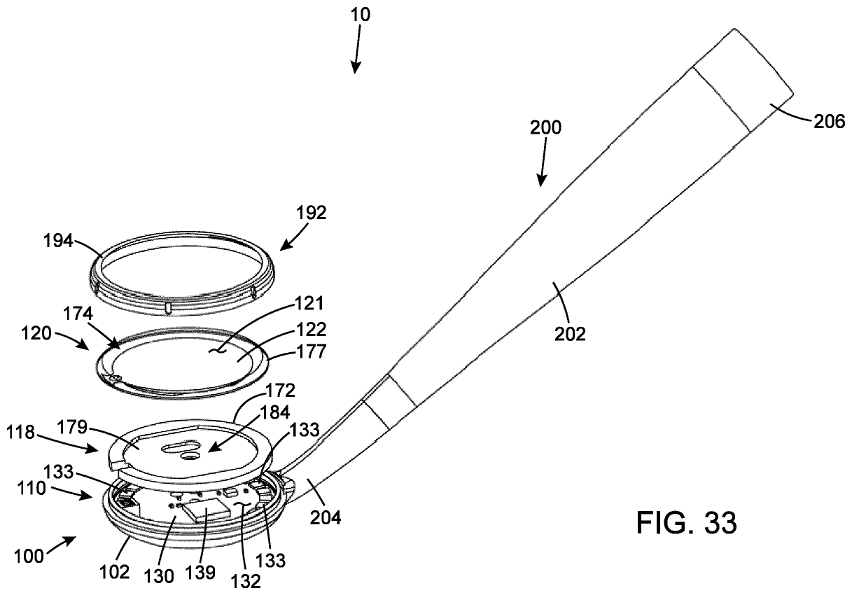

Referring to FIGS. 32 and 33, when the optical diffusing assembly 118 is present as a single diffusing layer, the optical diffusing assembly 118 can be substantially circular and be made of the same diffusing material throughout. In the implementation shown, the optical diffusing assembly 118 defines an additional layer receiving surface 179. In some implementations, the additional layer receiving surface 179 can be configured to receive thereon a heating layer 182 extending between the optical diffusing assembly 118 and the reflective layer 120. It is to be noted that another type of layer can also be received on the additional layer receiving surface 179. In some implementations, it can be an additional layer made of a material having different optical properties compared to the remainder of the optical diffusing assembly 118. For instance, it can be a layer made of a material that has a significantly reduced ability to transmit light emitted from the light sources 133 compared to the remainder of the optical diffusing assembly 118. In some implementations, examples of this type of material can include a light-blocking material. In some implementations, the additional layer can be made of a reflective material. In some implementations, the additional layer can be made of a reflective material that is light-blocking and reflective. Providing such additional layer can contribute to directing the diffused light produced by the optical diffusing assembly 118 toward the diffused light guiding channel 174 rather than enabling a portion of the diffused light to be “lost” toward a center region of the reflective layer 120. When the additional is made of a reflective material, the reflective material can prevent a portion of the diffused light from passing therethrough by reflecting it, for instance in a preferred direction.

As mentioned above, in FIGS. 32 and 33, the optical diffusing assembly 118 can be substantially circular. In other implementations, the optical diffusing assembly 118 can be shaped as a ring configured for placement against the diffused light guiding channel 174. In other words, the center region of the optical diffusing assembly 118, which can optionally include an additional layer receiving surface, can be omitted.

In some implementations and as shown in FIGS. 29 and 31, the light sources 133 can be positioned directly underneath the diffused light guiding channel 174, with the optical diffusing assembly 118 in between, such that the light emitted from the light sources 133 can successively travel directly through the optical diffusing assembly 118 and through the diffused light guiding channel 174. In other words, the light sources 133 and the diffused light guiding channel 174 share a corresponding common transversal axis 189 extending normally, relative to the housing bottom wall 104, across the optical diffusing assembly 118. FIG. 31 illustrates an example of such common transversal axis 189, which is shown extending upwardly from one of the light sources 133, across the optical diffusing assembly 118, and then across the thickness of the transparent layer 177 within the diffused light guiding channel 174. This configuration can be advantageous when compared to light sources that would be provided in a center region of the head portion 100 and that would thus have to travel outwardly toward the outside of the reflective layer 120 to reach the diffused light guiding channel 174. It is to be understood that although the diffused light guiding channel 174 is schematized as being substantially straight for illustrative purposes, i.e., with borders on each side, diffused light can nonetheless travel outwardly, in particular when the width of the diffused light guiding channel 174 is defined by a reflective surface provided on the back of the transparent layer, as shown in FIG. 33 for instance.

In some implementations, the optical diffusing assembly 118 can be made of a single piece of a diffusing material, such as shown in FIGS. 32 and 33. In other implementations, the optical diffusing assembly 118 can be ring-shaped, i.e., annular, and can define a diffusing layer opening in a central region thereof. In such implementations, the optical diffusing layer can be coupled with an additional layer received within the diffusing layer opening of the optical diffusing assembly. The additional layer and the ring-shaped optical diffusing assembly can be coupled together with glue, or there can be a coupling feature between the two, such a male-female coupling. In such implementations, the role of the additional layer can be similar than as described above when the additional layer is received onto the additional layer receiving surface 179, i.e., to favor, concentrate or direct, the travelling of the diffused light toward the diffused light guiding channel 174 of the reflective layer 120 rather than toward the center region of the reflective layer 120, which can contribute to limiting light losses.

Referring to FIG. 33, in this example implementation, the optical diffusing assembly 118 also defines a fastener opening 184 that can receive a fastener 186 therein. The fastener 186 can be used to secure the optical diffusing assembly 118 to the housing bottom wall 104. FIGS. 4, 7 and 9 illustrate an example of a threaded hole defined in the housing bottom wall 104 that can receive a fastener.

In implementations where the optical diffusing assembly 118 includes an outer diffusing layer 140 and an intermediate diffusing layer 150, the outer diffusing layer 140 can be made of an outer diffusing layer material, and the intermediate diffusing layer 150 can be made of an intermediate diffusing layer material. The outer diffusing layer material and the intermediate diffusing layer material can be the same or different. In implementations where the intermediate diffusing layer material is different than the outer diffusing layer material, one of the intermediate diffusing layer material and the outer diffusing material can be less diffusing than the other, for instance to facilitate the travelling of the light emitted from the light sources 133 and retaining the light intensity of the emitted light therethrough. The first-pass diffused light travelling out the intermediate diffusing layer 150 can thus still have a relatively strong intensity, and then the first-pass diffused light can pass through the outer diffusing layer material to produce second-pass diffused light.

Thus, in the example illustrated in FIGS. 4 to 6, light emitted from the light sources 133 encounters a lower surface of the intermediate diffusing layer 150, and at least a portion of the light is transmitted through the intermediate diffusing layer 150. The at least a portion of the transmitted light exits the intermediate diffusing layer 150 through an upper surface of the intermediate diffusing layer 150. Light exiting the upper surface of the intermediate diffusing layer 150 encounters a lower surface of the outer diffusing layer 140, and at least a portion of the light is transmitted through the outer diffusing layer 150. Then, at least a portion of the transmitted light exits the outer diffusing layer 140 through an outer surface of the outer diffusing layer 140 as diffused light, including the outwardly oriented outer surface 156 of the optical diffusing assembly sidewall 154. It is to be understood that the terms “upper” and “lower” as used herein are relative terms, for the purpose of illustration. For reference, in the Figures, the intermediate diffusing layer 150 is considered to be placed lower than the outer diffusing layer 140.

Although not illustrated in the implementation shown in FIGS. 25 to 33, it is to be understood that the optical diffusing assembly 118 of this example implementation can also include a plurality of superposed diffusing layers, as mentioned above.

In the implementation shown in FIGS. 25 to 33, light emitted by the light sources 133 encounters a lower surface of the optical diffusing assembly 118, and at least a portion of the light is transmitted through the optical diffusing assembly 118. The at least a portion of the transmitted light through the optical diffusing assembly 118 exits the optical diffusing assembly 118 through an upper surface of the optical diffusing assembly 118 as diffused light. The diffused light then passes through the diffused light guiding channel 174 defined in the reflective layer 120. As mentioned above, the diffused light guiding channel 174 is defined through the thickness of the transparent layer 177 of the reflective layer 120, and the transparent layer 177 can be made of a transparent material such as glass or sapphire, for instance. It is to be understood that in some implementations, the transparent material of the diffused light guiding channel 174 does not contribute to producing the diffused light, but rather the diffused light produced by the optical diffusing assembly 118 is passing through the transparent material without being further diffused. In other implementations, the diffused light guiding channel 174 can be configured to produce diffused light as discussed in further detail below. The optical diffusing assembly 118 described herein, whether integrated in the reflective layer 120 or provided as an additional component in the head portion 100 of the dental instrument, is present to produce diffused light, which otherwise would not be produced by the presence of a transparent material only.

Turning back to FIGS. 4 to 6, in the implementation shown, the optical diffusing assembly 118 includes an upper portion 148 having an arcuate profile defined by the outwardly oriented outer surface 156. More particularly, in implementations where the optical diffusing assembly 118 includes an intermediate diffusing layer 140 and an outer diffusing layer 150, the outer diffusing layer 150 can include the upper portion 148 having the arcuate profile defined by the outwardly oriented outer surface 156. Providing such an arcuate profile may assist in providing a more diffused illumination. In other words, in the illustrated implementation, the outwardly oriented outer surface 156 of the upper portion 148 of the outer diffusing layer 150 is a convex surface. The upper portion 148 can be further defined by a concave surface 155 that curves inwardly, i.e., toward a center of the head portion 100. This concave surface 155 can thus be referred to as an inwardly oriented concave surface. The combination of the outwardly oriented outer surface 156 that is convex and the inwardly oriented concave surface together define an inwardly projecting edge 152.

In turn, the combination of the reflective layer 120, and more particularly the peripheral wall 124 of the reflective layer 120, and the inwardly projecting edge 152 together define a channel 180 having a width WG. As shown in FIGS. 4 to 6, the width WG of the channel 180 increases from a lowest point of the channel 180 to a top of the channel 180. Accordingly, the channel 180 is thus free from overhangs and/or undercuts. This configuration of the channel 180 can provide various benefits, such as facilitating sterilization of the head portion 100 using ultraviolet (UV) light, such as UV-C light, by enabling the UV light to contact the substantially entire surface of the inwardly oriented concave surface 155 given the absence of undercut areas that may otherwise create a shadow that would prevent the UV light from accessing a given area of the inwardly oriented concave surface 155. This configuration of the channel 180 can thus result in less time being required to perform sterilization of the head portion 100, and/or may result in fewer (or no) positional adjustments of the head portion 100 within an UV-C chamber during a sterilization cycle.

It is to be understood that although in FIGS. 4 to 6, the width WG of the channel 180 is shown as increasing from the lowest point of the channel 180 to the top of the channel 180, in other implementations, the width WG of the channel 180 can remain substantially constant.

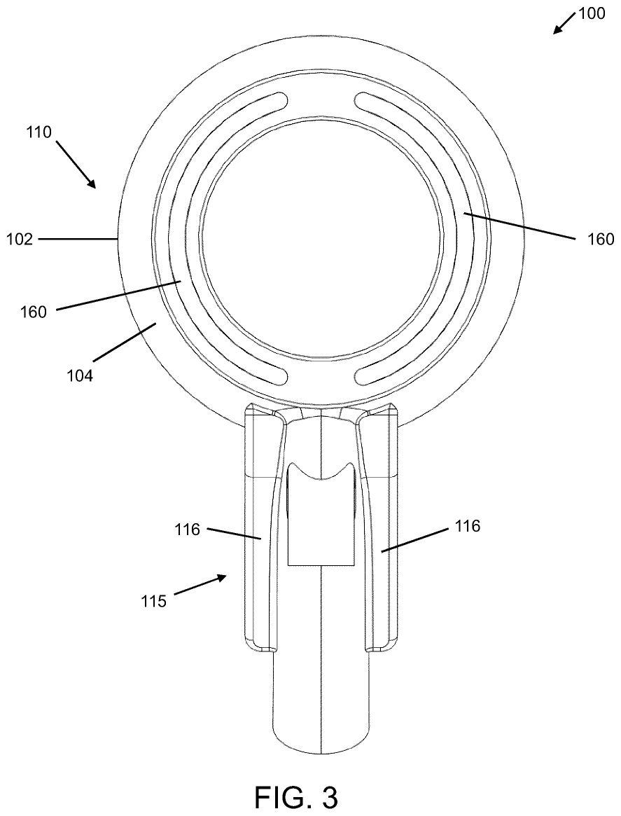

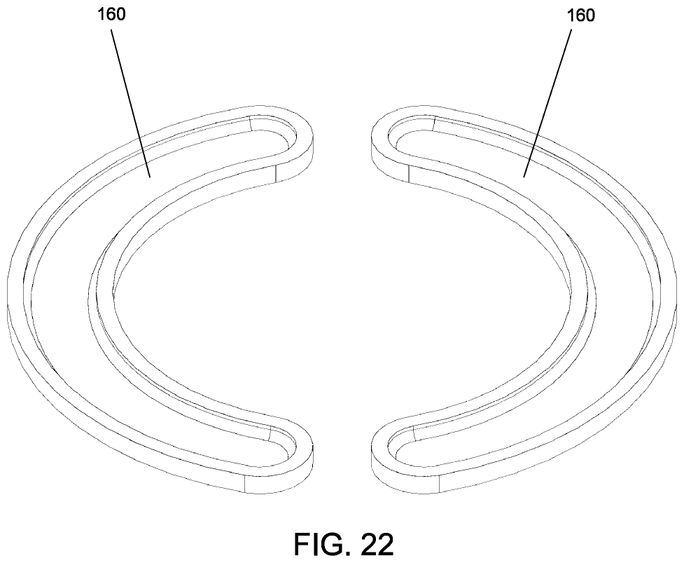

Referring to FIGS. 3, 7 and 22, the housing bottom wall 104 includes diffusing layer openings 112 configured to receive therein a corresponding optical diffusing layer 160. In the illustrated implementation, the housing bottom wall 104 includes two diffusing layer openings 112 and two optical diffusing layers 160. In this implementation, the housing bottom wall 104 is thus partially defined by the optical diffusing layers 160. The optical diffusing layers 160 are configured to diffuse the light emitted by the additional light sources 135. In some implementations, the optical diffusing layer 160 can be made of a diffusing layer material that is translucent. In some implementations, the optical diffusing layer 160 can be made of one or more of polystyrene, poly(methyl methacrylate), polycarbonate, and polypropylene, for instance.

### Light Source Distribution

With reference now to FIGS. 10 to 13 and 33, more details regarding the light sources and the light source distribution will be provided.

As used herein, the expression “light source distribution” refers to the distribution of the light sources within the light source receiving cavity of the head portion. An example of a light source distribution is a circumferential light source distribution or a peripheral light source distribution, as detailed below. In other implementations, the light source distribution can refer to a random distribution of the light sources. In yet other implementations, the light source distribution can refer to a distribution of the light sources that is according to a given pattern of the light sources.

FIGS. 10 to 13 and 33 illustrate an example of light sources 133 that can be received in the light source receiving cavity 116 of the open-top housing 110. In the implementation shown, the light sources 133 are a component of a printed circuit board (PCB) 130. In this particular example, the printed circuit board 130 includes the light sources 133 described above that are provided on an upper surface 132 of the printed circuit board 130, and also further includes additional light sources 135 that are provided on a lower surface 134 of the printed circuit board 130. It is to be noted that the additional light sources 135 can be optional. As for the light sources 133, the additional light sources 135 can include LEDs, while in other implementations, any other types of light sources that are configured to emit light can also be suitable.

In the illustrated implementations, the light sources 133 are distributed according to a light source distribution that can be referred to as a circumferential light source distribution, with at least some of the light sources 133 being aligned along a circular profile that is concentric with the substantially circular surface area of the housing bottom wall 104 of the open-top housing 110. In the circumferential light source distribution, the light sources 133 can optionally be distributed inwardly from the peripheral wall 124 of the reflective layer 120, as exemplified in FIGS. 4 to 6, 29 and 31. In the implementation shown, the light sources 133 are spaced-apart from one another, and adjacent ones of the light sources 133 are provided at a regular internal from one another. In the illustrated example, the circumferential light source distribution includes twenty-four (24) light sources 133. It is to be understood that more or fewer light sources can be included depending for instance on the choice of light sources, their illuminating performance, other constraints within the open-top housing 110 and with the optical diffusing assembly 118, and the intended use of the dental instrument, among other factors. Thus, when using the expression “circumferential light source distribution”, it is intended to mean that at least some of the light sources 133 are distributed along a circular profile that is concentric with the substantially circular surface area of the housing bottom wall 104 of the open-top housing 110, but that further light sources can be present either inwardly or outwardly of the circular profile, or both inwardly and outwardly from the circular profile. Furthermore, the expression “circumferential light source distribution” is used in the context of the housing sidewall 102 open-top housing 110 that is substantially cylindrical, with the housing bottom wall 104 having a substantially circular surface area. It is to be understood that when the open-top housing has a different shape, the expression “circumferential light source distribution” can be changed to a “peripheral light source distribution”.

Referring back to FIGS. 4 to 6, 29 and 31, the light sources 133 of the circumferential light source distribution can be distributed so as to be positioned inwardly from the peripheral wall 124 of the reflective layer 120. According to this example of circumferential light source distribution, the light sources 133 are therefore positioned underneath the reflective layer 120. When the light sources 133 are positioned underneath the reflective layer 120 and the optical diffusing assembly 118 is configured such as shown in FIGS. 4 to 6, the diffused light can travel outwardly from the peripheral wall 124 of the reflective layer 120, i.e., toward the exterior of the reflective layer 120. Alternatively, when the light sources 133 are positioned underneath the reflective layer 120 and the optical diffusing assembly 118 is configured such as shown in FIGS. 25 to 33 and more particularly FIGS. 29 and 31, the diffused light can travel through the diffused light guiding channel 174 defined in the reflective layer 120, inwardly of the peripheral wall 124 of the reflective layer 120.

In other implementations, the circumferential light source distribution can include inward light sources provided within the periphery of the circular profile defined by the outermost light sources 133 (not shown). The inward light sources can be distributed according to a given pattern, or they can be distributed randomly. Providing inward light sources can contribute to increasing the intensity of the emitted light that travels out to the optical diffusing assembly 118. Furthermore, although adjacent ones of the light sources 133 are shown as being provided at a regular internal from one another, in other implementations, the light sources 133 can be provided as two or more sets of light sources 133, with the sets of light sources 133 being provided at a given distance from each other. In some implementations, the light sources 133 provided on the upper face 132 of the printed circuit board 130 can be configured similarly to the configuration of the additional light sources 135 provided on the lower surface 134 of the printed circuit board 130.

In addition, although there can be various benefits to the circumferential light source distribution in which the outermost ones of the light sources 133 are provided inwardly from the peripheral wall 124 of the reflective layer 120 as described above, it is to be understood that in other implementations, some or all of the light sources 133 can be provided outwardly from the peripheral wall 124 of the reflective layer 120.

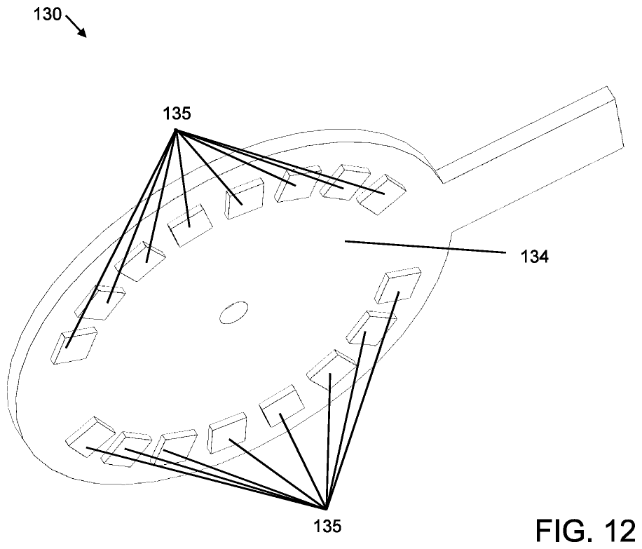

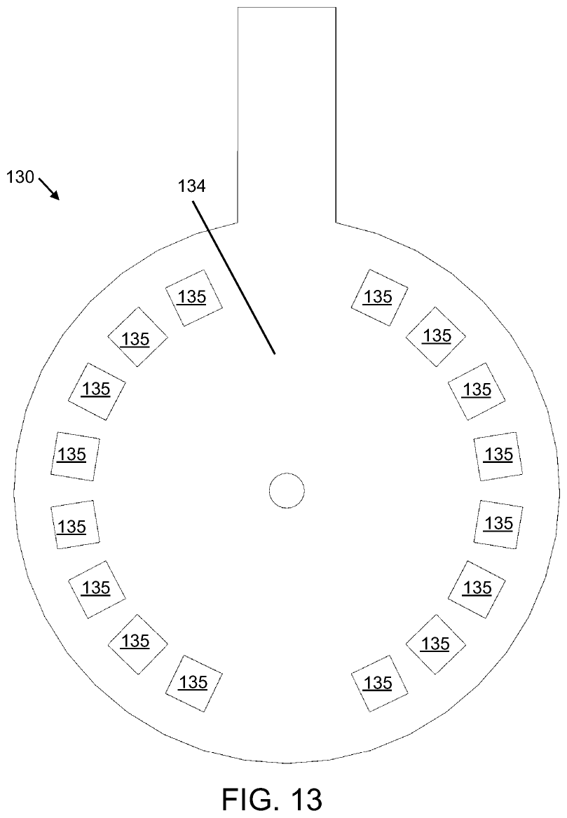

Referring to FIGS. 12 and 13 and as mentioned above, the printed circuit board 130 can further include additional light sources 135 that are provided on the lower surface 134 of the printed circuit board 130. In the implementation shown, the additional light sources 135 are provided as a first set of additional light sources 135 and a second set of additional light sources 135. The first and second sets of additional light sources 135 are aligned along a circular profile that is concentric with the substantially circular surface area of the housing bottom wall 104 of the open-top housing 110. The optical diffusing layers 160 are thus shaped as semicircular optical diffusing layers. The additional light sources 135 are configured to emit light in a direction that is opposite the outwardly oriented reflective surface 122 of the reflective layer 120. In other words, the additional light sources 135 are configured to emit light away, i.e., outwardly, from the housing bottom wall 104 of the open-top housing 110. In the example shown in FIGS. 12, 13 and 32, light emitted by the additional light sources 135 thus encounters an upper surface of a corresponding optical diffusing layer 160, and at least a portion of the light is transmitted through the corresponding optical diffusing layer 160 and exits the corresponding optical diffusing layer 160 through an outer surface thereof as diffused light. While in the illustrated implementation, sixteen (16) additional light sources 135 are illustrated, it will be appreciated that more or fewer additional light sources 135 be provided in one or more alternative implementations.

The presence of the additional light sources 135 can provide various benefits, such as enabling the health care provider to use the dental instrument 10 to retract the inner cheek of the patient's mouth by contacting the reflective surface 122 of the reflective layer 120 with the inner cheek, thereby exposing the housing bottom wall 104 and associated optical diffusing layers 160 received in the diffusing layer openings 112 so that the diffused light from the additional light sources 135 can illuminate the oral cavity of the patient's mouth.

It is to be noted that in alternative implementations (not shown), the head portion can be configured such that light sources are provided in the light source receiving cavity and positioned so as to illuminate in a direction opposite of the outwardly oriented reflective surface of the reflective layer, without the diffusing layer openings and an optical diffusing layer being present, also to provide light in the oral cavity of the patient's mouth while the health care provider uses the dental instrument to retract the inner cheek of the patient's mouth by contacting the reflective surface of the reflective layer with the inner cheek.

### Additional Features of the Optical Diffusing Assembly

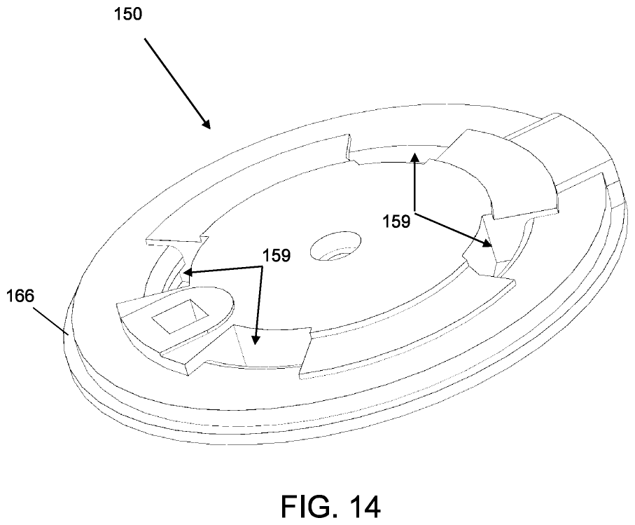

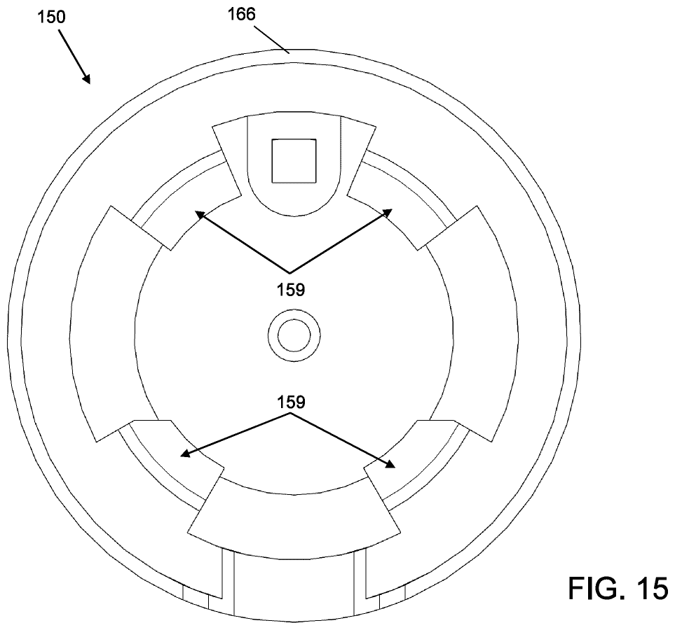

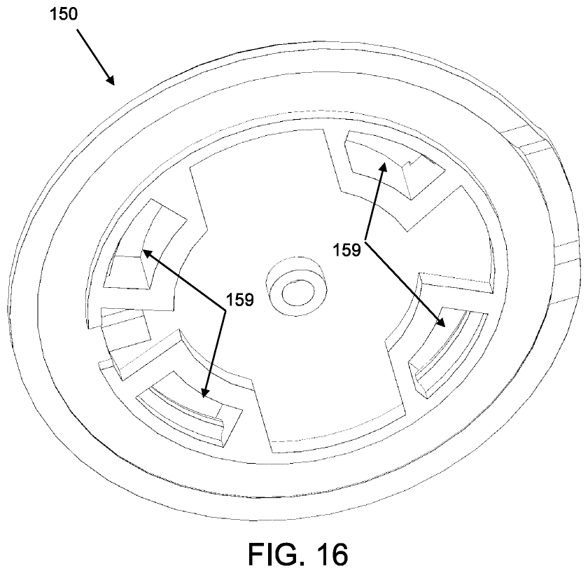

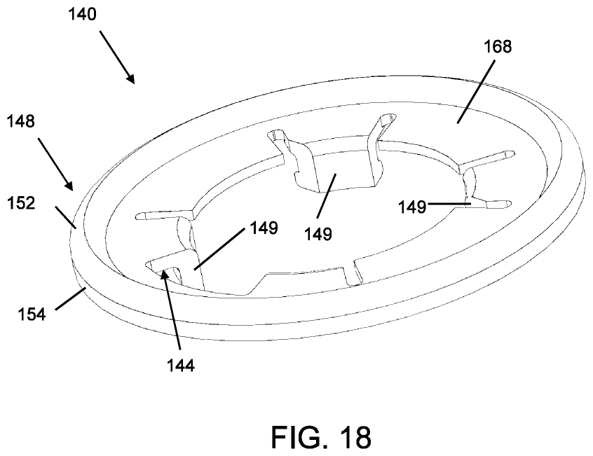

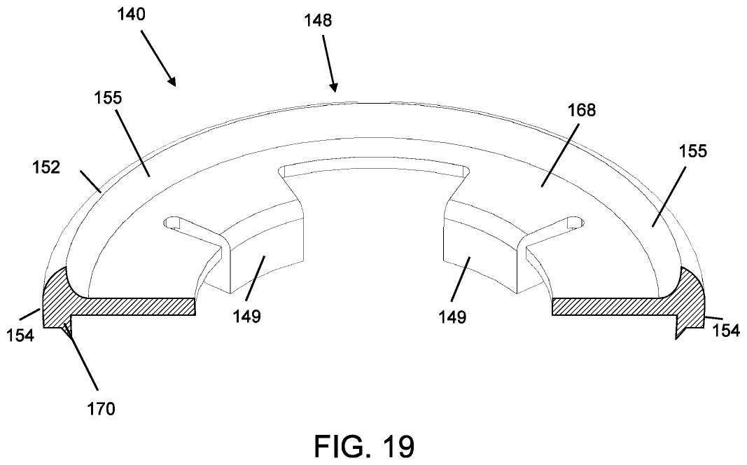

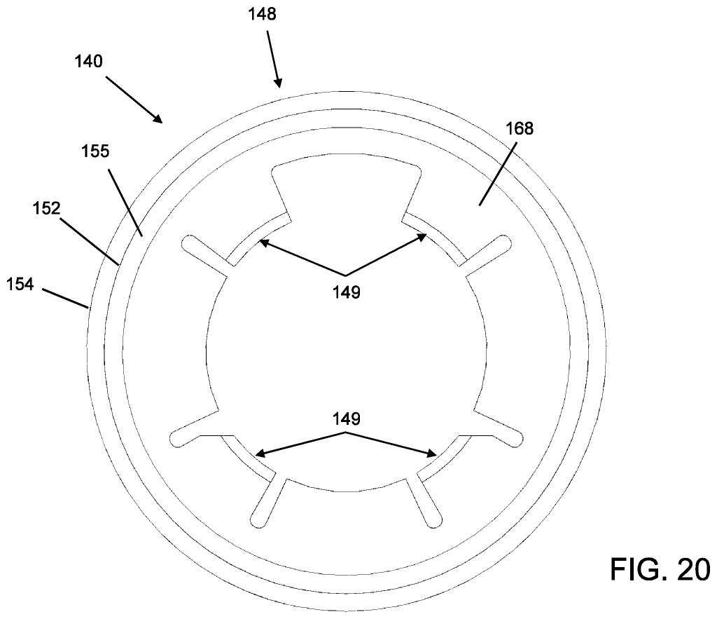

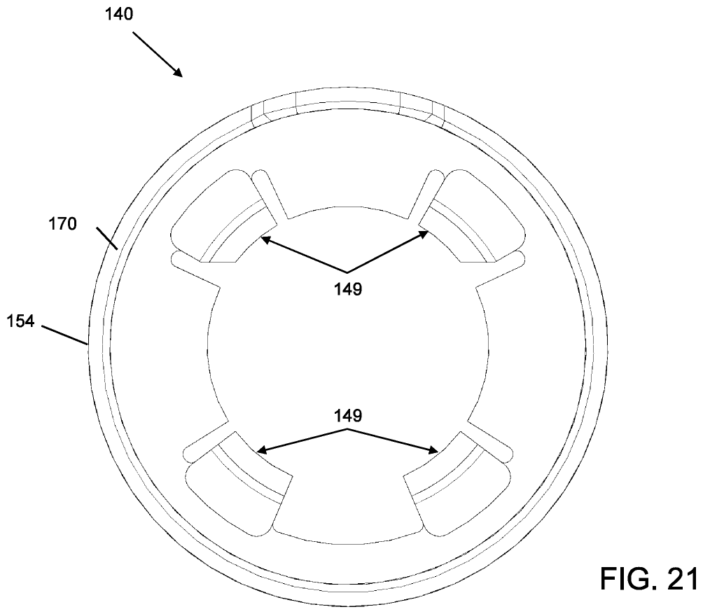

With reference to FIGS. 14 to 21, more details regarding an optical diffusing assembly that includes an intermediate diffusing layer and outer diffusing layer will be provided.

FIGS. 14 to 16 illustrate an example of an intermediate diffusing layer 150 that can form part of the optical diffusing assembly 118 as described herein. The intermediate diffusing layer 150 is shown as being substantially circular, and includes slots 159 distributed along a circular profile. The sidewall of the intermediate diffusing layer 150, forming part of the optical diffusing assembly sidewall 154, includes a shoulder 166.

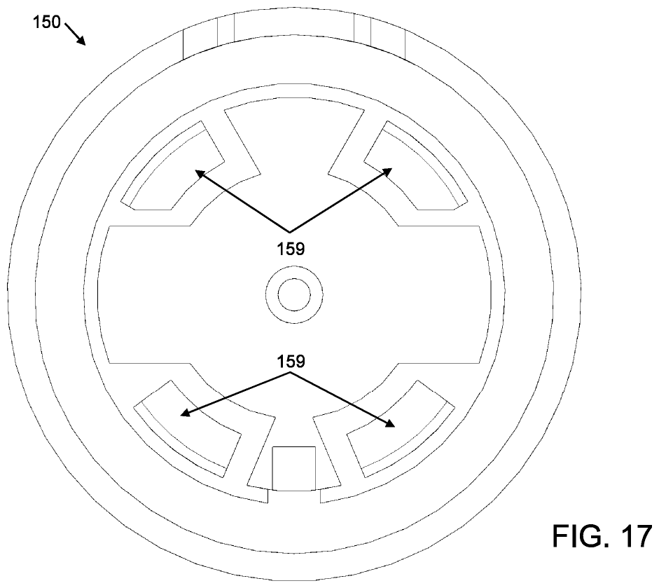

FIGS. 17 to 21 illustrate an example of an outer diffusing layer 140 that can form part of the optical diffusing assembly 118 described herein. The outer diffusing layer 140 is shown as being substantially annular. As described above, the outer diffusing layer 140 includes the outwardly oriented outer surface 156 that is convex and the inwardly oriented concave surface that together define an inwardly projecting edge 152. The outer diffusing layer 140 further includes a reflective layer receiving surface 168 configured to receive the reflective layer 120 thereon, as shown in FIGS. 4 to 6. The outer diffusing layer 140 also includes a downwardly projecting rim 170. The downwardly projecting rim 170 is configured to abut the shoulder 166 of the intermediate diffusing layer 150 when the outer diffusing layer 140 is positioned superposed to the intermediate diffusing layer 150, as shown in FIGS. 4 to 6. The outer diffusing layer 140 and the intermediate diffusing layer 150 can thus have complementary shapes to facilitate their superposition while enabling the formation of a gap therebetween if desired.

The intermediate diffusing layer 150 has an intermediate diffusing layer surface area and the outer diffusing layer 104 has an outer diffusing layer surface area, the outer diffusing layer surface area being smaller than intermediate diffusing layer surface area. In the implementation shown, the intermediate diffusing layer surface area is larger than the circular profile along which the light sources 133 are distributed. The intermediate diffusing layer surface area thus extends past the peripheral wall 124 of the reflective layer 120. Still in the implementation show, the intermediate diffusing layer surface area, at its largest, is sufficiently small to enable the intermediate diffusing layer 150 to be at least partially received in the light source receiving cavity 116.

In contrast, the outer diffusing layer surface area, at its largest, can be sufficiently large to extend outwardly such that the outwardly oriented outer surface 156 of the outer diffusing layer 140 coincides with the housing sidewall outer surface 108. This configuration can contribute to avoid the formation of shoulders or crests in proximity of the head portion sidewall 158, which in turn can have benefits when the dental instrument is subjected to sterilization using UV light. The outer diffusing layer 140 has an outer diffusing layer surface area that is sufficiently large to define at least a portion of the head portion sidewall 158 such that the head portion sidewall 158 is substantially continuous.

Accordingly, the optical diffusing assembly 118 shown in the implementation of FIGS. 4 to 6 includes both the intermediate diffusing layer 150 and the outer diffusing layer 140 is superposed to the light sources 133 and extends outwardly past outermost ones of the light sources 133, in a direction away from a center of the light source receiving cavity 116, so as to diffuse the light emitted by the light sources 133 and produce diffused light. This configuration of the optical diffusing assembly 118, and the presence of the optical diffusing assembly sidewall 154 forming part of the head portion sidewall 158, can enable the light emitted by the light sources 133 to travel upwardly and outwardly through the portion of the optical diffusing assembly 118 that extends between the peripheral wall 124 of the reflective layer 120 and the housing sidewall 102 to provide diffused light over a range of 180° from one side of the head portion 100 to the other side of the head portion 100. In some implementations, this configuration of the optical diffusing assembly 118 can produce a halo of diffused light around and outwardly of the reflective layer 120, the diffused light appearing substantially uniform at any location of the optical diffusing assembly 118. The diffused light can thus travel away from the outwardly oriented outer surface 156, in a normal direction therefrom. In some implementations, this effect can also be achieved when the optical diffusing assembly 116 is said to extend at least between the light sources 133, i.e., outwardly therefrom, and the housing sidewall outer surface 108.

The outer diffusing layer 140 further includes downwardly extending retaining tabs 149 configured to be engaged with the slots 159 defined in the intermediate diffusing layer 150. In the illustrated example, the downwardly extending retaining tabs 149 of the outer diffusing layer 140 are configured to be engaged with the complementary slots 159 of the intermediate diffusing layer 140 as a “press fit”. Accordingly, in the illustrated implementation, the outer diffusing layer 140 is configured to be releasably engageable with the intermediate diffusing layer 150. Such an arrangement can provide various benefits, such as enabling the reflective layer 120 and the outer diffusing layer 140 to be removable from the head portion 100 as a sub-assembly 190. It is to be noted that other types of engagements can also be suitable to couple the outer diffusing layer 140 and the intermediate diffusing layer 150 together.

It is to be understood that although the optical diffusing assembly 118 as described herein and as shown in the illustrated implementations are being provided in an upper portion of the head portion 100, in other implementations, an optical diffusing assembly 118 can be provided in a lower portion of the head portion 100, in a reverse configuration compared to the optical diffusing assembly 118 provided in the upper portion of the head portion 100. There can thus be a head portion 100 that includes a first and second optical diffusing assemblies 118 as described herein, or alternatively, the head portion 100 can include a single optical diffusing assembly 118 in the lower portion thereof. When the head portion 100 includes a single optical diffusing assembly 118 in the lower portion thereof, the optical diffusing layers 160 as described herein can be provided in the upper portion of the head portion 100.

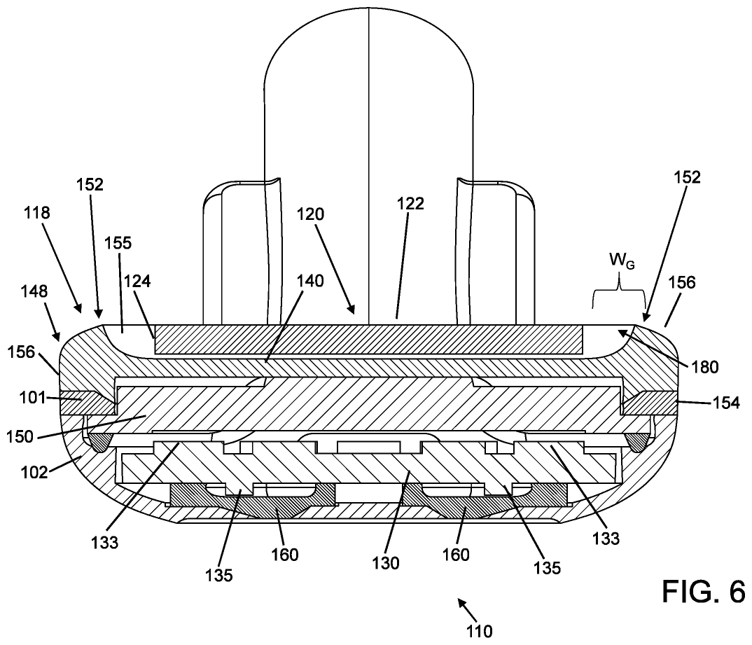

In some implementations, the thickness of either one of the outer diffusing layer 140 and the intermediate diffusing layer 150, or both, can be modified to achieve a desired degree of diffused light. In some implementations, the thickness of the outer diffusing layer 140, in an area that is superposed to the intermediate diffusing layer 150, can be thinner than the intermediate diffusing layer 150, as shown for instance in FIG. 6. In other implementations, the thickness of the outer diffusing layer 140, in an area that is superposed to the intermediate diffusing layer 150, can be thicker than the intermediate diffusing layer 150. In some implementations, the thickness of either one of the outer diffusing layer 140 and the intermediate diffusing layer 150, or both, can be determined at least in part according to the depth of the open-top housing 110 and the remaining space available once other components are introduced into the open-top housing 110.

As mentioned above and with reference to FIGS. 25 to 33, the optical diffusing assembly 118 can also refer to a single diffusing layer made of a diffusing material, the optical diffusing assembly 118 being superposed to the light sources and underneath the reflective layer 120 to produce diffused light that can travel across a diffused light guiding channel 174 defined in the reflective layer. Accordingly, the term “assembly” as used herein can be understood to mean that the optical diffusing assembly can include at least one diffusing layer, although the expression can also be used to designate an optical diffusing assembly that is integrated in the reflective layer, as described in further detail below.

### Alternative Implementations of the Optical Diffusing Assembly

In some implementations, the optical diffusing assembly can be integrated within the reflective layer 120, and the optical diffusing layer can thus be omitted. In such implementations, the reflective layer 120 can be configured to enable production of diffused light. In order to do so, the reflective layer 120 can include a portion having optical properties that are modified compared to the optical properties of the remaining of the reflective layer 120 to enable light emitted from the light sources to be scattered, i.e., diffused, when passing therethrough. When the reflective layer 120 is substantially circular, the portion of the reflective layer 120 that is configured to diffuse light can be ring-shaped and can extend circumferentially along the peripheral wall 124 of the reflective layer 120. In some implementations, the portion of the reflective layer 120 that enables diffusing light can correspond to the diffused light guiding channel 174. In other words, when the optical diffusing assembly does not include a distinct component corresponding to an optical diffusing layer such as described in FIGS. 1 to 33, the optical properties of the portion of the reflective layer 120 that corresponds to the diffused light guiding channel 174 can be modified compared to the optical properties of the remaining of the reflective layer 120, and in particular of the transparent layer 177, to enable the diffusion, i.e., the scattering, of light emitted from the light sources directly through the diffused light guiding channel 174.

Various techniques can be used to modify the optical properties of the reflective layer 120 at the determined location of the diffused light guiding channel 174 and enable production of diffused light. In some implementations, the portion of the reflective layer 120 where the diffused light guiding channel 174 is intended to be located can be subjected to a surface treatment. The surface treatment can be done on the top surface of the transparent layer and/or the bottom surface of the transparent layer. Examples of surface treatments can include a mechanical blasting of the glass surface, for instance using sand, glass beads or another abrasive material, to etch the glass surface so that light passing through this surface can be scattered. Alternatively, a chemical product, such as an acid, can be used to etch the glass and produce a similar effect. Another example of a surface treatment can include the application of a diffusing coating to the top surface of the transparent layer and/or the bottom surface of the transparent layer at the location of the diffused light guiding channel 174. In other implementations, the reflective layer 120 can be created using multiple layers of glass provided in an alternate configuration with one or more interlayers made of a diffusing material, at the location where the diffused light guiding channel 174 is intended to be located. In yet other implementations, the reflective layer 120 can include light diffusing agents, such as nano barium sulfate, calcium carbonate, and silica, at the location of the diffused light guiding channel 174. It is to be understood that any technique enabling the scattering of light through the diffused light guiding channel 174 can also be suitable.

In some implementations, the diffused light guiding channel 174 and the reflective layer 120 can be integral with each other, and the change in the optical properties of the reflective layer 120 at a desired location can result in the creation of the diffused light guiding channel 174 near the peripheral wall 124 of the reflective layer 120. In other implementations, the reflective layer 120 and the diffused light guiding channel 174 can be coupled with each other, for instance with an adhesive or any other suitable technique that enables the diffused light guiding channel 174 to remain in position with respect to the remaining of the reflective layer 120.

### Additional Features of the Dental Instrument

More details regarding additional features of the dental instrument will now be provided.

In some implementations, the head portion 100 can include an optical detector, such as a camera, configured to detect a characteristic of the diffused light or a characteristic of the ambient light. For instance, the characteristic of the diffused light can include an illuminance, a colour temperature, a color gradient, etc.

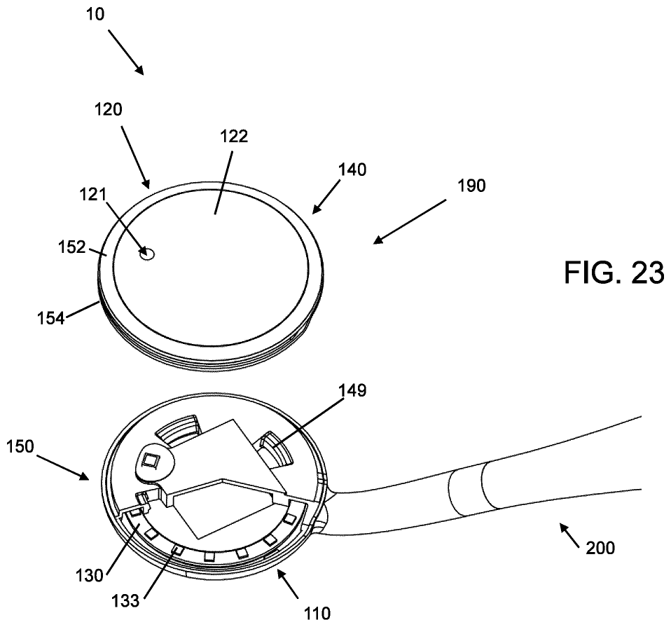

With reference to FIG. 23, in implementations where the optical detector is present and the reflective layer is present, at least a portion 121 of the reflective layer 120 can be a one-way mirror. A one-way mirror is a reciprocal mirror that appears reflective on one side and transparent at the other. The at least a portion 121 of the reflective layer can be strategically positioned superposed to the optical detector, and the optical detector can in turn be positioned onto the printed circuit board 130.

When the optical detector is present and configured to detect a characteristic of the diffused light or a characteristic of the ambient light, the detected characteristic of the diffused light or the detected characteristic of the ambient light can be compared a corresponding target value, and the light sources 133 and/or the additional light sources 135 can be configured to adjust a light output based on the comparison.

For example, in the implementation illustrated in FIGS. 10, 11 and 33, the printed circuit board 130 includes a logic chip 139 configured to monitor a characteristic of the diffused light or a characteristic of the ambient light, and automatically adjust the output of light sources 133 and/or the additional light sources 135 to promote a target illuminance and/or colour temperature within an oral cavity of a patient's mouth.

FIG. 23 is a partially exploded, partially cross-sectional, perspective view of the head portion 100 of the dental instrument 10 described herein, with the outer diffusing layer 140 and the reflective layer 120 shown separated from the head portion 100. As illustrated in FIG. 23, the outer diffusing layer 140, reflective layer 120 and gasket 101 can be configured to be removable from the head portion 100 as the sub-assembly 190 described above.

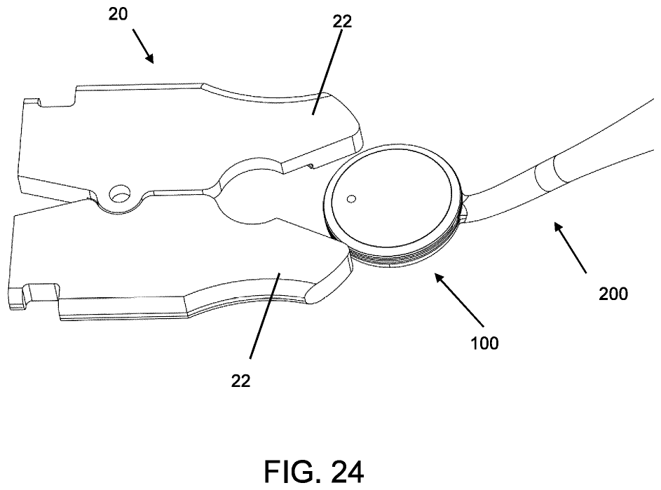

FIG. 24 is a perspective view of the head portion 100 described herein, and of a tool 20 configured to facilitate separation of the sub-assembly 190 that includes the outer diffusing layer 140, reflective layer 120 and gasket 101 from the head portion 100. The tool 20 is configured to facilitate the removal of the sub-assembly 190 from the head portion 100. This configuration can provide various benefits, such as enabling the replacement of at least the reflective layer 120. Indeed, such reflective layers used in the context of dental procedures can become scratched or damaged, so having the opportunity to easily replace a damaged component of the head portion 100 without having to replace the entire head portion 100 can save both costs and time.

The tool 20 includes a sub-assembly engaging tongs 22 configured to grasp the sub-assembly 190.

With reference to FIGS. 25 to 33, in some implementations, the reflective layer 120 can be removably engageable with the remainder of the components of the head portion 100 of the dental instrument 10. Referring more particularly to FIGS. 29, 31 and 33, the removable engagement of the reflective layer 120 with the remainder of the components of the head portion 100 of the dental instrument 10 can be done via a head portion ring 192. The head portion ring 192 can be screwable to a thread defined on the housing sidewall 102, although other types of engagement can also be suitable. For instance, the head portion ring 192 can be removably engaged with the open-top housing 110 via a snap fit or a press fit. As shown in FIGS. 29 and 31, the head portion ring 192 can include an inwardly extending projection 194 defining a reflective layer engaging surface 196 for engaging with the peripheral wall 124 of the reflective layer 120. In turn, the reflective layer 120 can be shaped as a truncated cone, i.e., the reflective layer can be a frustoconical reflective layer (or the transparent layer 177 can be a frustoconical transparent layer), with an outwardly extending slope 198 having an angle that is complementary to the angle of the inwardly extending projection of the head portion ring such that the outwardly extending slope 198 can abut the reflective layer engaging surface 196. Thus, the peripheral wall 124 of the reflective layer 120 can be configured to abut the reflective layer engaging surface 196 of the inwardly extending projection 194 of the head portion ring 194 that is removably engageable with the head portion 100, the reflective layer engaging surface 196 and the outwardly extending slope 198 having complimentary angles.

When the reflective layer is placed over the optical diffusing assembly 118, the head portion ring can be superposed thereto and via a rotation motion, the inwardly extending projection will eventually exert a downward pressure onto the outwardly extending slope of the reflective layer. This engagement of the head portion ring 192 and the reflective layer 120 can facilitate locking the reflective layer 120 in place onto the optical diffusing assembly 118 and with the remainder of the components of the head portion 100. Although in the illustrated implementation, the interaction of the peripheral wall 124 of the reflective layer 120 and the head portion ring 192 is achieved with the complementary angles of the outwardly extending slope 198 and the inwardly extending projection 194, respectively, it is to be understood that other types of interaction that enables securing the reflective layer 120 to the head portion 100 can also be suitable. In the illustrated implementation, a gasket 199, such as a compressible gasket, can be provided on a top edge of housing sidewall 102 such that when the head portion ring 192 is screwed in, the reflective layer 120 can press downwardly onto the gasket 199, thereby sealing the components of the dental instrument 10 provided in the open-top housing 110. In other words, the gasket 199 can be compressible such that when subjected to a downward force as the head portion ring 192 is being engaged with the open-top housing 110, the components of the dental instrument 10 provided in the open-top housing 110 can be sealed within the open-top housing 110. The gasket 199 can thus provide a waterproof and moisture-proof seal to prevent the components of the head portion 100 to be damaged, for instance during steam sterilization. Once again, depending on the interaction between the head portion ring 192 and the reflective layer 120, other alternatives enabling the water and moisture proofing of the components of the dental instrument 10 provided in the open-top housing 110 can also be envisioned.

In such implementations, the sub-assembly 190 can thus include the head portion ring 192 and the reflective layer 120, and optionally a heating layer if coupled with the reflective layer 120. When it is determined that the top surface of the reflective layer is damaged, and the health care provider wishes to replace the reflective layer 120, the head portion ring 192 can be disengaged from the housing sidewall 120 of the open-top housing 110, and the reflective layer 120 can be discarded. A replacement reflective layer can then be used and placed onto the optical diffusing assembly 118 and the head portion ring 192 can subsequently be screwed back on, or engaged with the open-top housing 110 according to another means. In some implementations, it may be desirable to change the gasket 199 as well if needed, which can be done concomitantly with the change of the reflective layer 120. Similarly, in some implementations, it may be desirable to change the optical diffusing assembly 118 if needed, which can also be done concomitantly with the change of the reflective layer 120. In some implementations, the gasket 199 can be elastic to facilitate its disengagement from the open-top housing 110.

With such a configuration of the open-top housing 110, the head portion ring 192 and the gasket 199, the head portion 100 of the dental instrument 10, as well as other portions of the dental instrument 10 not discussed in further detail herein, can be subjected to steam sterilization, for instance in an autoclave, without moisture and water infiltrating within the head portion 100 and without pressurized steam damaging the components of the head portion 100.

FIG. 33 is a partially exploded perspective view of the head portion 100 of the dental instrument 10 as described herein, with the optical diffusing assembly 118 and the reflective layer 120 shown separated from the head portion 100. As illustrated in FIG. 33, the optical diffusing assembly 118, the reflective layer 120 and optionally the gasket 199 can be configured to be removable from the head portion 100 as the sub-assembly 190 described above.

Referring to FIGS. 29 and 31, in some implementations, the open-top housing 110 can include a bottom reflective layer 195 coupled to the housing bottom wall 104. In such implementations, the housing bottom wall 104 can include a reflective layer receiving cavity 193 configured to receive the bottom reflective layer 195 therein. In the implementation shown, the bottom reflective layer 195 is inserted into the reflective layer receiving cavity 193 such that the outer surface of the bottom reflective layer is substantially flush with the housing bottom wall 104 located outwardly of the bottom reflective layer 195. The housing bottom wall 104 can thus include a step change at the transition between the housing bottom wall 104 located outwardly of the bottom reflective layer 195 and the reflective layer receiving cavity 193.

In some implementations, the bottom reflective layer 193 can be glued to the housing bottom wall 104, and a gasket 191 can be provided between the peripheral wall of the bottom reflective layer 195 and the step change defined in the housing bottom wall 104. As mentioned for the gasket 199 described above, this gasket 191 can also provide a waterproof and moisture-proof seal to prevent the components of the head portion 100 to be damaged, for instance during steam sterilization. The bottom reflective layer 195 can thus be inserted into the reflective layer receiving cavity 193 to compress the gasket 191 onto the step change of the housing bottom wall 104. The dental instrument 10 described herein can thus include two reflective layers, one on each side of the head portion 100.

It will be appreciated that for simplicity and clarity of illustration, where considered appropriate, reference numerals may be repeated among the figures to indicate corresponding or analogous elements. In addition, numerous specific details are set forth in order to provide a thorough understanding of the example implementations described herein. However, it will be understood by those of ordinary skill in the art that the example implementations described herein may be practiced without these specific details. In other instances, well-known methods, procedures, and components have not been described in detail so as not to obscure the example implementations described herein. Also, the description is not to be considered as limiting the scope of the example implementations described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

While the above description describes features of example implementations, it will be appreciated that some features and/or functions of the described implementations are susceptible to modification without departing from the spirit and principles of operation of the described implementations. For example, the various characteristics which are described by means of the represented implementations or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred implementations and examples, but should be given the broadest interpretation consistent with the description as a whole.

1. A dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
a reflective layer having a peripheral wall and comprising a diffused light guiding channel extending peripherally along the peripheral wall and inwardly thereof;
an open-top housing comprising a housing bottom wall and a housing sidewall together defining a light source receiving cavity;
light sources received within the light source receiving cavity and configured to emit light; and
an optical diffusing assembly superposed to the light sources and provided underneath the diffused light guiding channel, the optical diffusing assembly being configured to diffuse the light emitted by the light sources and produce the diffused light.

2. The dental instrument of claim 1, wherein the light sources are spaced-apart from one another and positioned according to a light source distribution.

3. The dental instrument of claim 2, wherein the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

4. The dental instrument of any one of claims 1 to 3, wherein the light sources are provided at a regular interval from one another.

5. The dental instrument any one of claims 1 to 4, wherein the head portion further comprises a printed circuit board comprising the light sources.

6. The dental instrument of any one of claims 1 to 5, wherein the light sources comprise light-emitting diodes.

7. The dental instrument of any one of claims 1 to 6, wherein the optical diffusing assembly is directly superposed to the light sources and the diffused light guiding channel of the reflective layer is directly superposed to the optical diffusing assembly such that the light sources and the diffused light guiding channel share a corresponding common transversal axis extending normally relative to the housing bottom wall across the optical diffusing assembly.

8. The dental instrument of any one of claims 1 to 7, wherein the reflective layer comprises a transparent layer and a reflective surface positioned underneath the transparent layer, the optical diffusing assembly being positioned closest to the reflective surface.

9. The dental instrument of any one of claims 1 to 8, wherein the optical diffusing assembly comprises a single diffusing layer.

10. The dental instrument of any one of claims 1 to 8, wherein the optical diffusing assembly comprises a plurality of diffusing layers.

11. The dental instrument of any one of claims 1 to 10, wherein the optical diffusing assembly is sized to be contained within an outer periphery defined by the peripheral wall of the reflective layer.

12. The dental instrument of claim 11, wherein the diffused light guiding channel coincides with the outer periphery defined by the peripheral wall.

13. The dental instrument of claim 11, wherein the diffused light guiding channel is provided at a distance from the outer periphery defined by the peripheral wall, thereby defining a gap therebetween.

14. The dental instrument of any one of claims 1 to 13, wherein the diffused light guiding channel extends continuously at least from a 1 o'clock location to an 11 o'clock location.

15. The dental instrument of any one of claims 1 to 13, wherein the reflective layer further defines an optical detector opening at a 12 o'clock location.

16. The dental instrument of claim 15, wherein the optical detector is configured to:
detect at least one of an illuminance and a colour temperature;
compare at least one of:
a detected illuminance with a target illuminance, and
a detected colour temperature with a target colour temperature; and
adjust a light output of the light sources based on the at least one comparison.

17. The dental instrument of any one of claims 1 to 16, wherein the optical diffusing assembly is substantially circular.

18. The dental instrument of any one of claims 1 to 17, wherein the optical diffusing assembly is ring-shaped and defines a diffusing layer opening.

19. The dental instrument of claim 18, further comprising an additional layer configured to be received in the diffusing layer opening.

20. The dental instrument of any one of claims 1 to 17, wherein the optical diffusing assembly defines an additional layer receiving surface configured to receive an additional layer thereon.

21. The dental instrument of claim 19 or 20, wherein the additional layer is a heating layer.

22. The dental instrument of claim 19 or 20, wherein the additional layer has a reduced ability to transmit light emitted by the light sources compared to a remainder of the optical diffusing assembly.

23. The dental instrument of claim 19 or 20, wherein the additional layer is made of a light-blocking material.

24. The dental instrument of claim 19 or 20, wherein the additional layer is made of a reflective material.

25. The dental instrument of any one of claims 20 to 24, wherein the optical diffusing assembly defines a fastener opening configured to receive a fastener therein to secure the optical diffusing assembly to the housing bottom wall.

26. The dental instrument of any one of claims 1 to 25, wherein the optical diffusing assembly is made of a diffusing material comprising one or more of polystyrene, poly(methyl methacrylate), polycarbonate, and polypropylene.

27. The dental instrument of claim 26, wherein the diffusing material is configured to transmit between 25% and 90% of light having a wavelength between 400 nm and 800 nm.

28. The dental instrument of claim 26 or 27, wherein the diffusing material has a density ranging between 1 000 kg/m³ and about 1 300kg/m³.

29. The dental instrument of any one of claims 1 to 28, wherein the optical diffusing assembly has a half-power angle ranging between about 1° and about 55°.

30. The dental instrument of any one of claims 1 to 29, wherein the optical diffusing assembly has a diffusing layer thickness ranging between about 0.5 mm and about 7 mm.

31. The dental instrument of any one of claims 1 to 28, wherein the optical diffusing assembly has a diffusing layer thickness of about 1 mm and a half-power angle of the optical diffusing assembly is between about 1° and about 5°.

32. The dental instrument of any one of claims 1 to 28, wherein the optical diffusing assembly has a diffusing layer thickness of about 2 mm and the half-power angle of the optical diffusing assembly is between about 18° and about 30°.

33. The dental instrument of any one of claims 1 to 28, wherein the optical diffusing assembly has a diffusing layer thickness of about 3 mm and the half-power angle of the optical diffusing assembly is between about 30° and about 45°.

34. The dental instrument of any one of claims 1 to 28, wherein the optical diffusing assembly has a diffusing layer thickness of about 4 mm and the half-power angle of the optical diffusing assembly is between about 40° and about 55°.

35. The dental instrument of any one of claims 1 to 34, wherein the head portion further comprises a head portion ring engageable with the open-top housing.

36. The dental instrument of claim 35, wherein the head portion ring is screwable to a thread defined on the housing sidewall.

37. The dental instrument of claim 35 or 36, wherein the head portion ring comprises an inwardly extending projection defining a reflective layer engaging surface for engaging with the peripheral wall of the reflective layer.

38. The dental instrument of claim 37, wherein the reflective layer is shaped as a frustoconical reflective layer defining an outwardly extending slope configured to abut the reflective layer engaging surface of the inwardly extending projection of the head portion ring.

39. The dental instrument of any one of claims 35 to 38, further comprising a gasket provided on a top edge of the housing sidewall, the gasket being compressible when subjected to a downward force as the head portion ring is being engaged with the open-top housing, thereby sealing the components of the dental instrument provided in the open-top housing.

40. The dental instrument of any one of claims 35 to 39, wherein the head portion ring and the reflective layer, and optionally the gasket, are configured to be removable from the head portion as a sub-assembly.

41. The dental instrument of claim 40, wherein the reflective layer is a replaceable reflective layer.

42. The dental instrument of any one of claims 1 to 41, wherein the head portion further comprises an additional optical diffuser assembly, the additional optical diffuser assembly comprising:
additional light sources oriented opposite to the light sources; and
an additional optical diffusing assembly defining at least part of the housing bottom wall of the open-top housing.

43. The dental assembly of claim 42, wherein the additional optical diffusing assembly comprises a diffusing layer received into diffusing layer openings defined in the housing bottom wall.

44. The dental instrument of claim 42 or 43, wherein the additional light sources comprise light-emitting diodes.

45. The dental instrument of any one of claims 1 to 44, wherein the housing bottom wall defines a reflective layer receiving cavity, and the head portion further comprises a bottom reflective layer received in the reflective layer receiving cavity.

46. The dental instrument of claim 45, wherein the housing bottom wall defines a step change at a transition between the housing bottom wall located outwardly of the bottom reflective layer and the reflective layer receiving cavity.

47. The dental instrument of claim 46, further comprising a gasket between a peripheral wall of the bottom reflective layer and the step change defined in the housing bottom wall.

48. A dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
a reflective layer having an outwardly oriented reflective surface;
an open-top housing comprising a housing sidewall and a housing bottom wall defining a diffusing layer opening, the housing bottom wall and the housing sidewall together defining a light source receiving cavity;
light sources received within the light source receiving cavity and configured to emit light in a direction opposite the outwardly oriented reflective surface of the reflective layer; and
an optical diffusing layer having an inwardly oriented surface facing the light sources and being received into the diffusing layer opening of the housing bottom wall, the optical diffusing layer being configured to diffuse the light emitted by the light sources and produce the diffused light.

49. The dental instrument of claim 48, wherein the light sources are spaced-apart from one another and positioned according to a light source distribution.

50. The dental instrument of claim 49, wherein the housing sidewall is substantially cylindrical and the housing bottom wall has a substantially circular surface area, and the light source distribution is a circumferential light source distribution, with at least some of the light sources being aligned along a circular profile that is concentric with the substantially circular surface area of the open-top housing.

51. The dental instrument of any one of claims 48 to 50, wherein the optical diffusing layer comprises a first optical diffusing layer and a second optical diffusing layer and the diffusing layer opening comprises a first diffusing layer opening and a second diffusing layer opening, the first optical diffusing layer being received in the first diffusing layer opening and the second optical diffusing layer being received in the second diffusing layer opening, the first and second optical diffusing layers being shaped as semicircular optical diffusing layers.

52. The dental instrument any one of claims 48 to 51, wherein the head portion further comprises a printed circuit board comprising the light sources.

53. The dental instrument of any one of claims 48 to 52, wherein the light sources comprise light-emitting diodes.

54. The dental instrument of any one of claims 48 to 53, wherein the optical diffusing assembly is made of a diffusing material comprising one or more of polystyrene, poly(methyl methacrylate), polycarbonate, and polypropylene.

55. The dental instrument of claim 54, wherein the diffusing material is configured to transmit between 25% and 90% of light having a wavelength between 400 nm and 800 nm.

56. The dental instrument of claim 54 or 55, wherein the diffusing material has a density ranging between 1 000 kg/m³ and about 1 300kg/m³.

57. The dental instrument of any one of claims 48 to 56, wherein the optical diffusing assembly has a half-power angle ranging between about 1° and about 55°.

58. The dental instrument of any one of claims 48 to 57, wherein the optical diffusing assembly has a diffusing layer thickness ranging between about 0.5 mm and about 7 mm.

59. The dental instrument of any one of claims 48 to 56, wherein the optical diffusing assembly has a diffusing layer thickness of about 1 mm and a half-power angle of the optical diffusing assembly is between about 1° and about 5°.

60. The dental instrument of any one of claims 48 to 56, wherein the optical diffusing assembly has a diffusing layer thickness of about 2 mm and the half-power angle of the optical diffusing assembly is between about 18° and about 30°.

61. The dental instrument of any one of claims 48 to 56, wherein the optical diffusing assembly has a diffusing layer thickness of about 3 mm and the half-power angle of the optical diffusing assembly is between about 30° and about 45°.

62. The dental instrument of any one of claims 48 to 56, wherein the optical diffusing assembly has a diffusing layer thickness of about 4 mm and the half-power angle of the optical diffusing assembly is between about 40° and about 55°.

63. The dental instrument of any one of claims 48 to 62, wherein the head portion further comprises an optical detector, the optical detector being configured to:
detect at least one of an illuminance and a colour temperature;
compare at least one of:
a detected illuminance with a target illuminance, and
a detected colour temperature with a target colour temperature; and
adjust a light output of the light sources based on the at least one comparison.

64. The dental instrument of any one of claims 48 to 63, wherein the housing bottom wall defines a reflective layer receiving cavity, and the head portion further comprises a bottom reflective layer received in the reflective layer receiving cavity.

65. The dental instrument of claim 64, wherein the housing bottom wall defines a step change at a transition between the housing bottom wall located outwardly of the bottom reflective layer and the reflective layer receiving cavity.

66. The dental instrument of claim 65, further comprising a gasket between a peripheral wall of the bottom reflective layer and the step change defined in the housing bottom wall.

67. A system for use with a head portion of a dental instrument, the system comprising:
a reflective layer comprising:
a frustoconical transparent layer comprising a top surface, a bottom surface opposite the top surface, and a peripheral wall defining an outwardly extending slope; and
a reflective surface adjacent to the transparent layer;
wherein the peripheral wall of the reflective layer is configured to abut a reflective layer engaging surface of an inwardly extending projection of a head portion ring removably engageable with an open-top housing of the head portion, the reflective layer engaging surface and the outwardly extending slope having complimentary angles.

68. The system of claim 67, wherein the reflective layer comprises a diffused light guiding channel extending along the peripheral wall of the transparent layer, inwardly thereof, the diffused light guiding channel being configured to enable passage of diffused light therethrough.

69. The system of claim 67, further comprising a gasket positionable underneath the reflective layer and configured to seal a remainder of the components of the head portion when a downward pressure is applied to the reflective layer.

70. The system of claim 68, wherein the gasket is a compressible gasket.

71. A dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
an open-top housing comprising a housing sidewall and a housing bottom wall defining a reflective layer receiving cavity, the housing sidewall and the housing bottom wall together defining a housing cavity;
a top reflective layer at least partially received within the open-top housing; and
a bottom reflective layer received in the reflective layer receiving cavity;
wherein the top reflective layer and the bottom reflective layer are configured such that their respective reflective surfaces are opposite to each other.

72. The dental instrument of claim 71, wherein the housing bottom wall defines a step change at a transition between the housing bottom wall located outwardly of the bottom reflective layer and the reflective layer receiving cavity.

73. The dental instrument of claim 72, further comprising a gasket between a peripheral wall of the bottom reflective layer and the step change defined in the housing bottom wall.

74. The dental instrument of any one of claims 71 to 73, wherein the top reflective layer comprises:
a frustoconical transparent layer comprising a top surface, a bottom surface opposite the top surface, and a peripheral wall defining an outwardly extending slope; and
a reflective surface adjacent to the transparent layer.

75. The dental instrument of claim 74, wherein the head portion comprises a head portion ring configured to be removably engageable with the open-top housing.

76. The dental instrument of claim 75, wherein the head portion ring comprises an inwardly extending projection defining a reflective layer engaging surface for engaging with the peripheral wall of the top reflective layer.

77. The dental instrument of claim 75, wherein the peripheral wall of the top reflective layer is configured to abut the reflective layer engaging surface of the inwardly extending projection of the head portion ring, the reflective layer engaging surface and the outwardly extending slope having complimentary angles.

78. The system of any one of claims 74 to 77, wherein the top reflective layer comprises a diffused light guiding channel extending along the peripheral wall of the transparent layer, inwardly thereof, the diffused light guiding channel being configured to enable passage of diffused light therethrough.

79. The system of claim 78, further comprising a gasket positionable underneath the top reflective layer and configured to seal a remainder of the components of the head portion when a downward pressure is applied to the top reflective layer.

80. The system of claim 79, wherein the gasket is a compressible gasket.

81. A dental instrument for illuminating an oral cavity of a patient's mouth with diffused light, the dental instrument comprising:
a handle portion comprising an elongated member enabling the dental instrument to be handled; and
a head portion positioned at one longitudinal end of the elongated member, the head portion comprising:
an open-top housing comprising a housing bottom wall and a housing sidewall together defining a light source receiving cavity;
light sources received within the light source receiving cavity and configured to emit light; and
a reflective layer having a peripheral wall and comprising a diffused light guiding channel superposed to the light sources and extending peripherally along the peripheral wall and inwardly thereof, the diffused light guiding channel comprising:
an optical diffusing assembly configured to diffuse the light emitted by the light sources and produce the diffused light.
